(19) United States
(12) Patent Application Publication
(10) Pub. No.: US 2010/0087464 A1
(43) Pub. Date: Apr. 8, 2010

(54) PROTEIN KINASE INHIBITORS AND METHODS FOR USING THEREOF

(75) Inventors: Yuan Mi, Shanghai (CN); Pamela A. Albaugh, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton, BM (BM)

(21) Appl. No.: 12/443,626

(22) PCT Filed: Aug. 27, 2007
(86) PCT No.: PCT/US07/76871

§ 371 Date: Nov. 19, 2009

Related U.S. Application Data
(60) Provisional application No. 60/850,361, filed on Oct. 6, 2006.

(51) Int. Cl. A61K 31/505; C07D 249/08; A61K 31/4196; C12N 5/00; C07D 401/14; A61K 31/454; A61K 31/4439; C07D 403/14; A61K 31/4178; C07D 235/04; A61K 31/4184; C07D 271/06; A61K 31/4245; A61P 35/00; A61P 29/00; A61P 3/10; A61P 37/04

(52) U.S. Cl. 514/275; 548/266.4; 514/383; 435/375; 546/201; 514/323; 546/272.4; 514/339; 548/312.1; 514/397; 548/305.1; 514/394; 548/131; 514/364; 544/331

(57) ABSTRACT

The invention provides compounds and pharmaceutical compositions thereof, which are useful as protein kinase inhibitors, and methods for using such compounds to treat, ameliorate or prevent a condition associated with abnormal or deregulated kinase activity. In some embodiments, the invention provides methods for using such compounds to treat, ameliorate or prevent diseases or disorders that involve abnormal activation of TrkA, TrkB, TrkC, Abl, Bcr-Abl, cSrc, TPR-Met, Tie2, MET, FGFR3, Aurora, Axl, Bmx, BTK, c-kit, CHK2, Flt3, MST2, p70S6K, PDGFR, PKB, PKCα, Raf, ROCK-II, Rsk1, and SGK kinases, or a combination thereof.

### CROSS-REFERENCE TO RELATED APPLICATIONS

This application claims the benefit of U.S. provisional application Ser. No. 60/850,361, filed on Oct. 6, 2006, which is hereby incorporated by reference in its entirety.

### TECHNICAL FIELD

The invention relates to protein kinase inhibitors, and methods of using such compounds.

### BACKGROUND ART

The protein kinases represent a large family of proteins, which play a central role in the regulation of a wide variety of cellular processes and maintaining control over cellular function. A partial, non-limiting, list of these kinases include: receptor tyrosine kinases such as platelet-derived growth factor receptor kinase (PDGFR), the nerve growth factor receptor, TrkB, Met, and the fibroblast growth factor receptor, FGFR-3; non-receptor tyrosine kinases such Abl and the fusion kinase Bcr-Abl, Lck, Csk, Fes, Bmx and Src; and serine/threonine kinases such as B-Raf, C-Raf, Sgk, MAP kinases (e.g., MKK4, MKK6, etc.) and SAPK2α, SAPK2β and SAPK3. Aberrant kinase activity has been observed in many disease states including benign and malignant proliferative disorders, as well as diseases resulting from inappropriate activation of the immune and nervous systems.

### DISCLOSURE OF THE INVENTION

The invention provides compounds and pharmaceutical compositions thereof, which may be useful as protein kinase inhibitors.

In one aspect, the present invention provides compounds of Formula (1):

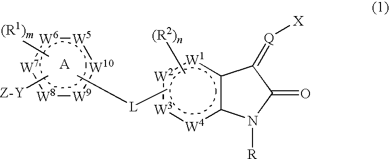

or pharmaceutically acceptable salts and tautomers thereof, wherein:

W¹, W², W³, W⁴, W⁵, W⁶, W⁷, W⁸, W⁹ and W¹⁰ are independently C or N; provided each of W¹, W², W³, W⁴, W⁵, W⁶, W⁷, W⁸, W⁹ and W¹⁰ is C when attached to L, Y, R¹ and R²;

Q is N, NNR, NO or CR⁰;

L is a bond, —O—, —NRC(O)—, —NRC(O)NR—, —C(O)NR—, —NR— or S;

R⁰, R¹ and R² are independently halo; C₁₋₆alkyl, C₂₋₆ alkenyl, or C₃₋₆ alkynyl, each of which may be optionally halogenated or optionally substituted with N, O or S; or an optionally substituted aryl, heteroaryl, carbocyclic ring or heterocyclic ring; or R⁰ is H;

each R is H or C₁₋₆alkyl;

X and Z are independently an optionally substituted aryl, heteroaryl, heterocyclic ring or carbocyclic ring;

Y is an optionally substituted heteroaryl;

alternatively, Ring A together with Y may form a fused heteroaryl; or Y and Z together may form a fused heteroaryl;

m is 0-4; and

n is 0-3;

provided said compound is not 3-(1H-pyrrol-2-ylmethylene)-6-{3-[3-(3-trifluoromethyl-phenyl)-[1,2,4]oxadiazol-5-yl]-phenylamino}-1,3-dihydro-indol-2-one.

In the above Formula (1), each of W¹, W², W³, W⁴, W⁵, W⁶, W⁷, W⁸, W⁹ and W¹⁰ may be C. In some examples, W¹, W², W³ and W⁴ are each C, and at least one of W⁵, W⁶, W⁷, W⁸, W⁹ and W¹⁰ is N. In other examples, two of W⁵, W⁶, W⁷, W⁸, W⁹ and W¹⁰ are N. In particular examples, R¹ and R² are independently halo, or an optionally halogenated C₁₋₆ alkyl or C₁₋₆ alkoxy. In some examples, m is 1 and n is 0.

In one embodiment, the invention provides compounds having Formula (2):

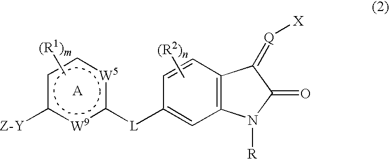

wherein R¹ and R² are independently halo, or an optionally halogenated C₁₋₆ alkyl or C₁₋₆ alkoxy;

W⁵ and W⁹ are independently C or N; provided each of W⁵ and W⁹ is C when attached to R¹;

X and Y are independently an optionally substituted heteroaryl;

Z is an optionally substituted aryl or heteroaryl;

alternatively, Ring A together with Y may form a fused heteroaryl; or Y and Z together may form a fused heteroaryl; and

m and n are independently 0-2.

In the above Formula (1) and (2), X and Y may independently be an optionally substituted a 5-6 membered heteroaryl having N, O or S. For example, X and Y may independently an optionally substituted pyrrolyl, imidazolyl, triazolyl, tetrazolyl, pyridyl, pyrimidinyl, oxazolyl, isoxazolyl, pyrazolyl, furanyl or oxadiazolyl. In particular examples, X is an optionally substituted pyrrolyl or imidazolyl. In other examples, Y is imidazolyl, triazolyl, pyrazole or oxadiazolyl. In yet other examples, Ring A together with Y form benzimidazolyl.

In the above Formula (1) and (2), Z may be an optionally substituted 5-7 membered aryl or heteroaryl. For example, Z may be an optionally substituted phenyl, pyridyl or furanyl. In other examples, Y and Z together form benzimidazolyl.

In the above Formula (1) and (2), L may be a bond or NH. In some examples, Q is CR⁰ and R⁰ is H or C₁₋₆alkyl.

In the above Formula (1) and (2), each X, Y and Z may optionally be substituted with halo, an optionally halogenated C₁₋₆ alkyl or C₁₋₆ alkoxy, wherein a carbon may be substituted with heteroatom selected from N, O or S; —C(O)NR³R⁴, —C(O)NR(CR₂)ₖ NR³R⁴, (CR₂)ₖCO₂R³, (CR₂)ₖCN, —NRS(O)₀₋₂R³, —S(O)₀₋₂ NR³R⁴, —NRS(O)₀₋₂ NR³R⁴, C(O)NR(CR₂)ₖOR³ or R⁵;

R³ and R⁴ are independently H, C₁₋₆ alkyl, C₃₋₇ cycloalkyl, or a 5-10 membered heterocyclic ring, aryl, or heteroaryl ring; or R³ and R⁴ together with N in NR³R⁴ form an optionally substituted ring;

R⁵ is C₃₋₇ cycloalkyl, 5-10 membered heterocyclic ring, aryl, or heteroaryl ring;

k is 0-4;

each R is H or C₁₋₆ alkyl.

In some examples, X may be optionally substituted with an optionally halogenated C₁₋₆ alkyl or C₁₋₆ alkoxy, —C(O)NR³R⁴, —C(O)NR(CR₂)ₖ NR³R⁴, (CR₂)ₖCO₂R³ or (CR₂)ₖCN, wherein R, R³ and R⁴ are independently H or C₁₋₆ alkyl; or R³ and R⁴ together with N in NR³R⁴ may form an optionally substituted ring, such as piperidinyl. In other examples, Z may be optionally substituted with C₁₋₆ alkyl, a halogenated C₁₋₆ alkyl (e.g., CF₃) or halo.

In another aspect, the invention provides pharmaceutical compositions comprising a therapeutically effective amount of a compound having Formula (1) or (2), and a pharmaceutically acceptable carrier.

The invention also provides methods for inhibiting kinases, comprising administering to a system or a subject in need thereof, a therapeutically effective amount of a compound having Formula (1) or (2), or pharmaceutically acceptable salts or pharmaceutical compositions thereof, thereby inhibiting said kinase. In one embodiment, the invention provides methods for inhibiting TrkA, TrkB, TrkC, Abl, Bcr-Abl, cSrc, TPR-Met, Tie2, MET, FGFR3, Aurora, Axl, Bmx, BTK, c-kit, CHK2, Flt3, MST2, p70S6K, PDGFR, PKB, PKCα, Raf, ROCK-II, Rsk1 or SGK kinases, or a combination thereof. More particularly, the invention provides methods for inhibiting Trk kinases, such as TrkA, TrkB, TrkC, or a combination thereof.

The invention also provides methods for using compounds having Formula (1) or (2) to treat, ameliorate or prevent a condition associated with abnormal or deregulated kinase activity. In one embodiment, the invention provides methods for treating a condition mediated by TrkA, TrkB, TrkC, Abl, Bcr-Abl, cSrc, TPR-Met, Tie2, MET, FGFR3, Aurora, Axl, Bmx, BTK, c-kit, CHK2, Flt3, MST2, p70S6K, PDGFR, PKB, PKCα, Raf, ROCK-II, Rsk1 or SGK kinase, or a combination thereof, comprising administering to a system or subject in need of such treatment an effective amount of a compound having Formula (1) or (2), or pharmaceutically acceptable salts or pharmaceutical compositions thereof, thereby treating said kinase-mediated condition. More particularly, the invention provides methods for treating a condition mediated by a Trk kinase, such as TrkA, TrkB, TrkC, or a combination thereof.

Examples of conditions which may be treated using the compounds of the invention include but are not limited to a cell proliferative disorder such as neuroblastoma, or a tumor or cancer of the breast, prostate or pancreas. In particular embodiments, the compounds of the invention may be used to treat prostate cancer or pancreatic cancer. The compounds of the invention may also be used to treat chronic pain, bone pain, abnormal angiogenesis, arthritis, diabetes, diabetic retinopathy, macular degeneration or psoriasis.

In another aspect, the invention provides the use of compounds having Formula (1) or (2), or pharmaceutically acceptable salts or pharmaceutical compositions thereof, for inhibiting a kinase, such as TrkA, TrkB, TrkC, Abl, Bcr-Abl, cSrc, TPR-Met, Tie2, MET, FGFR3, Aurora, Axl, Bmx, BTK, c-kit, CHK2, Flt3, MST2, p70S6K, PDGFR, PKB, PKCα, Raf, ROCK-II, Rsk1 or SGK kinase, or a combination thereof. In one embodiment, the invention provides the use of compounds having Formula (1) or (2), or pharmaceutically acceptable salts or pharmaceutical compositions thereof, for inhibiting Trk kinases, such as TrkA, TrkB, TrkC, or a combination thereof.

Furthermore, the invention provides the use of compounds having Formula (1) or (2), or pharmaceutically acceptable salts or pharmaceutical compositions thereof, in the manufacture of a medicament for treatment of a condition mediated by a kinase, such as TrkA, TrkB, TrkC, Abl, Bcr-Abl, cSrc, TPR-Met, Tie2, MET, FGFR3, Aurora, Axl, Bmx, BTK, c-kit, CHK2, Flt3, MST2, p70S6K, PDGFR, PKB, PKCα, Raf, ROCK-II, Rsk1 or SGK kinase, or a combination thereof. In one embodiment, the invention provides the use of compounds having Formula (1) or (2), or pharmaceutically acceptable salts or pharmaceutical compositions thereof, in the manufacture of a medicament for treatment of a condition mediated by Trk kinases, such as TrkA, TrkB, TrkC, or a combination thereof.

In the above methods for using the compounds of the invention, a compound having Formula (1) or (2) may be administered to a system comprising cells or tissues. In other embodiments, a compound having Formula (1) or (2) may be administered to a human or animal subject.

### MODES OF CARRYING OUT THE INVENTION

In one aspect, the present invention provides compounds of Formula (1):

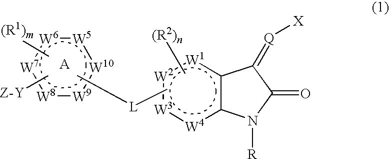

or pharmaceutically acceptable salts and tautomers thereof, wherein:

W¹, W², W³, W⁴, W⁵, W⁶, W⁷, W⁸, W⁹ and W¹⁰ are independently C or N; provided each of W¹, W², W³, W⁴, W⁵, W⁶, W⁷, W⁸, W⁹ and W¹⁰ is C when attached to L, Y, R¹ and R²;

Q is N, NNR, NO or CR⁰;

L is a bond, —O—, —NRC(O)—, —NRC(O)NR—, —C(O)NR—, —NR— or S;

R⁰, R¹ and R² are independently halo; C₁₋₆alkyl, C₂₋₆ alkenyl, or C₃₋₆ alkynyl, each of which may be optionally halogenated or optionally substituted with N, O or S; or an optionally substituted aryl, heteroaryl, carbocyclic ring or heterocyclic ring; or R⁰ is H;

each R is H or C₁₋₆alkyl;

X and Z are independently an optionally substituted aryl, heteroaryl, heterocyclic ring or carbocyclic ring;

Y is an optionally substituted heteroaryl;

alternatively, Ring A together with Y may form a fused heteroaryl; or Y and Z together may form a fused heteroaryl;

m is 0-4; and

n is 0-3;

provided said compound is not 3-(1H-pyrrol-2-ylmethylene)-6-{3-[3-(3-trifluoromethyl-phenyl)-[1,2,4]oxadiazol-5-yl]-phenylamino}-1,3-dihydro-indol-2-one.

In one embodiment, the invention provides compounds having Formula (2):

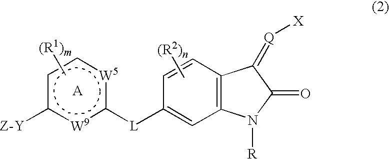

wherein R¹ and R² are independently halo, or an optionally halogenated C₁₋₆ alkyl or C₁₋₆ alkoxy;

W⁵ and W⁹ are independently C or N; provided each of W⁵ and W⁹ is C when attached to R¹;

X and Y are independently an optionally substituted heteroaryl;

Z is an optionally substituted aryl or heteroaryl;

alternatively, Ring A together with Y may form a fused heteroaryl; or Y and Z together may form a fused heteroaryl; and

m and n are independently 0-2.

In the above Formula (1) and (2), other moieties for R⁰, R¹ and R² may be used that are within the knowledge of those skilled in the art, including but not limited to OR, cyano, amino, amido, guanidino, ureayl, nitro and other inorganic substituents, etc.

Compounds having Formula (1) and (2) may be useful as protein kinase inhibitors. For example, compounds having Formula (1) or (2), and pharmaceutically acceptable salts, solvates, N-oxides, prodrugs and isomers thereof, may be used for the treatment of a kinase-mediated condition or disease, such as diseases mediated by TrkA, TrkB, TrkC, Abl, Bcr-Abl, cSrc, TPR-Met, Tie2, MET, FGFR3, Aurora, Axl, Bmx, BTK, c-kit, CHK2, Flt3, MST2, p70S6K, PDGFR, PKB, PKCα, Raf, ROCK-II, Rsk1 or SGK kinase, or a combination thereof.

The compounds of the invention may also be used in combination with a second therapeutic agent, for ameliorating a condition mediated by a protein kinase, such as a Trk-mediated condition. For example, the compounds of the invention may be used in combination with a chemotherapeutic agent to treat a cell proliferative disorder, including but not limited to, neuroblastoma, or a tumor or cancer of the breast, prostate or pancreas.

Examples of chemotherapeutic agents which may be used in the compositions and methods of the invention include but are not limited to anthracyclines, alkylating agents (e.g., mitomycin C), alkyl sulfonates, aziridines, ethylenimines, methylmelamines, nitrogen mustards, nitrosoureas, antibiotics, antimetabolites, folic acid analogs (e.g., dihydrofolate reductase inhibitors such as methotrexate), purine analogs, pyrimidine analogs, enzymes, podophyllotoxins, platinum-containing agents, interferons, and interleukins. Particular examples of known chemotherapeutic agents which may be used in the compositions and methods of the invention include, but are not limited to, busulfan, improsulfan, piposulfan, benzodepa, carboquone, meturedepa, uredepa, altretamine, triethylenemelamine, triethylenephosphoramide, triethylenethiophosphoramide, trimethylolomelamine, chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, dacarbazine, mannomustine, mitobronitol, mitolactol, pipobroman, aclacinomycins, actinomycin F(1), anthramycin, azaserine, bleomycin, cactinomycin, carubicin, carzinophilin, chromomycin, dactinomycin, daunorubicin, daunomycin, 6-diazo-5-oxo-1-norleucine, doxorubicin, epirubicin, mitomycin C, mycophenolic acid, nogalamycin, olivomycin, peplomycin, plicamycin, porfiromycin, puromycin, streptonigrin, streptozocin, tubercidin, ubenimex, zinostatin, zorubicin, denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, fluorouracil, tegafur, L-asparaginase, pulmozyme, aceglatone, aldophosphamide glycoside, aminolevulinic acid, amsacrine, bestrabucil, bisantrene, carboplatin, cisplatin, defofamide, demecolcine, diaziquone, elformithine, elliptinium acetate, etoglucid, etoposide, flutamide, gallium nitrate, hydroxyurea, interferon-alpha, interferon-beta, interferon-gamma, interleukin-2, lentinan, lonidamine, mitoguazone, mitoxantrone, mopidamol, nitracrine, pentostatin, phenamet, pirarubicin, podophyllinic acid, 2-ethylhydrazide, procarbazine, razoxane, sizofuran, spirogermanium, paclitaxel, tamoxifen, teniposide, tenuazonic acid, triaziquone, 2,2′,2″-trichlorotriethylamine, urethane, vinblastine, vincristine, and vindesine.

### DEFINITIONS

“Alkyl” refers to a moiety and as a structural element of other groups, for example halo-substituted-alkyl and alkoxy, and may be straight-chained or branched. An optionally substituted alkyl, alkenyl or alkynyl as used herein may be optionally halogenated (e.g., CF₃), or may have one or more carbons that is substituted or replaced with a heteroatom, such as NR, O or S (e.g., —OCH₂CH₂O—, alkylthiols, thioalkoxy, alkylamines, etc).

“Aryl” refers to a monocyclic or fused bicyclic aromatic ring containing carbon atoms. For example, aryl may be phenyl or naphthyl. “Arylene” means a divalent radical derived from an aryl group.

“Heteroaryl” as used herein is as defined for aryl above, where one or more of the ring members are a heteroatom. Examples of heteroaryls include but are not limited to pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, etc.

A “carbocyclic ring” as used herein refers to a saturated or partially unsaturated, monocyclic, fused bicyclic or bridged polycyclic ring containing carbon atoms, which may optionally be substituted, for example, with ═O. Examples of carbocyclic rings include but are not limited to cyclopropyl, cyclobutyl, cyclopentyl, cyclohexyl, cyclopropylene, cyclohexanone, etc.

A “heterocyclic ring” as used herein is as defined for a carbocyclic ring above, wherein one or more ring carbons is a heteroatom. For example, a heterocyclic ring may contain N, O, S, —N═, —S—, —S(O), —S(O)₂—, or —NR— wherein R may be hydrogen, C₁₋₄alkyl or a protecting group. Examples of heterocyclic rings include but are not limited to morpholino, pyrrolidinyl, pyrrolidinyl-2-one, piperazinyl, piperidinyl, piperidinylone, 1,4-dioxa-8-aza-spiro[4.5]dec-8-yl, etc.

Unless otherwise indicated, when a substituent is deemed to be “optionally substituted,” it is meant that the substituent is a group that may be substituted with one or more group(s) individually and independently selected from, for example, an optionally halogenated alkyl, alkenyl, alkynyl, alkoxy, alkylamine, alkylthio, alkynyl, amide, amino, including mono- and di-substituted amino groups, aryl, aryloxy, arylthio, carbonyl, carbocyclic, cyano, cycloalkyl, halogen, heteroalkyl, heteroalkenyl, heteroalkynyl, heteroaryl, heterocyclic, hydroxy, isocyanato, isothiocyanato, mercapto, nitro, O-carbamyl, N-carbamyl, O-thiocarbamyl, N-thiocarbamyl, C-amido, N-amido, S-sulfonamido, N-sulfonamido, C-carboxy, O-carboxy, perhaloalkyl, perfluoroalkyl, silyl, sulfonyl, thiocarbonyl, thiocyanato, trihalomethanesulfonyl, and the protected compounds thereof. The protecting groups that may form the protected compounds of the above substituents are known to those of skill in the art and may be found in references such as Greene and Wuts, Protective Groups in Organic Synthesis, 3^{rd} Ed., John Wiley & Sons, New York, N.Y., 1999, and Kocienski, Protective Groups, Thieme Verlag, New York, N.Y., 1994, which are incorporated herein by reference in their entirety.

The terms “co-administration” or “combined administration” or the like as used herein are meant to encompass administration of the selected therapeutic agents to a single patient, and are intended to include treatment regimens in which the agents are not necessarily administered by the same route of administration or at the same time.

The term “pharmaceutical combination” as used herein refers to a product obtained from mixing or combining active ingredients, and includes both fixed and non-fixed combinations of the active ingredients. The term “fixed combination” means that the active ingredients, e.g. a compound of Formula (1) and a co-agent, are both administered to a patient simultaneously in the form of a single entity or dosage. The term “non-fixed combination” means that the active ingredients, e.g. a compound of Formula (1) and a co-agent, are both administered to a patient as separate entities either simultaneously, concurrently or sequentially with no specific time limits, wherein such administration provides therapeutically effective levels of the active ingredients in the body of the patient. The latter also applies to cocktail therapy, e.g. the administration of three or more active ingredients.

“Mutant forms of BCR-Abl” means single or multiple amino acid changes from the wild-type sequence. Over 22 mutations have been reported to date with the most common being G250E, E255V, T315I, F317L and M351T.

“NTKR1” is the gene name equivalent to TrkA protein; “NTKR2” is the gene name equivalent to TrkB protein; and “NTKR3” is the gene name equivalent to TrkC protein.

The term “therapeutically effective amount” means the amount of the subject compound that will elicit a biological or medical response in a cell, tissue, organ, system, animal or human that is being sought by the researcher, veterinarian, medical doctor or other clinician.

The term “administration” and or “administering” of the subject compound should be understood to mean as providing a compound of the invention including a pro-drug of a compound of the invention to the individual in need of treatment.

Pharmacology and Utility

Compounds of the invention may modulate the activity of kinases and, as such, are useful for treating diseases or disorders in which kinases contribute to the pathology and/or symptomology of the disease. Examples of kinases that may be inhibited by the compounds and compositions described herein and against which the methods described herein may be useful include, but are not limited to, TrkA, TrkB, TrkC, Abl, Bcr-Abl, cSrc, TPR-Met, Tie2, MET, FGFR3, Aurora, Axl, Bmx, BTK, c-kit, CHK2, Flt3, MST2, p70S6K, PDGFR, PKB, PKCα, Raf, ROCK-II, Rsk1, and SGK kinases.

The Trk family of neurotrophin receptors (TrkA or “NTKR1”; TrkB or “NTKR2”; TrkC or “NTKR3”) is able to control tumor cell growth and survival as well as differentiation, migration and metastasis. The signaling pathway downstream of the Trk receptors involves the cascade of MAPK activation through the Shc, activated Ras, ERK-1 and ERK-2 genes, and the PLC-gamma transduction pathway (Sugimoto et al., Jpn J Cancer Res. 2001, 92: 152-60).

There is evidence that Trk tyrosine kinases play a role in the development of a variety of cancers including, for example, breast and prostate cancer. (Guate et al., Expression of p75LNGFR and Trk Neurotrophin Receptors in Normal and Neoplastic Human Prostate, BJU Int. 1999, 84:495 502; Tagliabue et al., J. Biol. Chem. 2000, 275:5388 5394.) Further, there is evidence that mediation of the Trk kinase signaling will provide beneficial biological effects. (LeSauteur et al., Adv. Behav. Biol. 1998, 49:615 625; Zhu et al., (1999) J. Clin. Oncology, 1999, 17:2419 28; Friess et al., Annals of Surgery 1999, 230:615-24.)

NTRK3 (TrkC) and its closely related family members NTRK1 (TrkA) and NTRK2 (TrkB) are implicated in the development and progression of cancer, possibly by upregulation of either the receptor, their ligand (nerve growth factor, brain derived neurotrophic factor, neurotrophins) or both (Rubin et al., Cancer Treat. Res. 2003, 115:1-18; Nakagawara, Cancer Lett. 2001, 169:107-14). High expression of NTRK2 and/or its ligand BDNF has been shown in pancreatic and prostate carcinomas, Wilm's tumors and neuroblastomas. In addition, high expression of NTRK3 is a hallmark of melanoma, especially in cases with brain metastasis. In many cases, high Trk expression is associated with aggressive tumor behavior, poor prognosis and metastasis.

NTKR2 (TrkB) protein is expressed in neuroendocrine-type cells in the small intestine and colon, in the alpha cells of the pancreas, in the monocytes and macrophages of the lymph nodes and of the spleen, and in the granular layers of the epidermis. Expression of the TrkB protein has been associated with an unfavorable progression of Wilms tumors and of neuroblastomas. Moreover, TrkB is expressed in cancerous prostate cells but not in normal cells.

NTRK2 is a potent inhibitor of anoikis, defined as apoptosis induced by loss of attachment of a cell to its matrix. By activating the phosphatidylinositol-3-kinase/protein kinase B signaling axis, NTRK2 was shown to promote the survival of non-transformed epithelial cells in 3-dimensional cultures and to induce tumor formation and metastasis of those cells in immuno-compromised mice.

Genetic abnormalities, i.e. point mutations and chromosomal rearrangements involving both NTRK2 and NTRK3, have been found in a variety of cancer types. In a kinome-wide approach to identify point mutants in tyrosine kinases, both NTRK2 and NTRK3 mutations were found in cell lines and primary samples from patients with colorectal cancer (Bardelli et al., Science 2003, 300:949), implicating the Trk family members in regulating metastasis and suggesting their functional relevance in colorectal cancer.

In addition, chromosomal translocations involving both NTRK1 and NTRK3 have been found in several different types of tumors. Gene rearrangements involving NTRK1 and a set of different fusion partners (TPM3, TPR, TFG) are a hallmark of a subset of papillary thyroid cancers (PTC) (Tallini, Endocr. Pathol. 2002, 13:271-88). Moreover, secretory breast cancer, infant fibrosarcoma and congenital mesoblastic nephroma have been shown to be associated with a chromosomal rearrangement t(12;15) generating a ETV6-NTRK3 fusion gene that was shown to have constitutive kinase activity, and transforming potential in several different cell lines including fibroblasts, hematopoietic cells and breast epithelial cells (Euhus et al., Cancer Cell 2002, 2:347-8; Tognon et al., Cancer Cell 2002, 2:367-76; Knezevich et al., Cancer Res. 1998, 58:5046-8; Knezevich et al., Nat. Genet. 1998, 18:184-7).

Abelson tyrosine kinase (i.e. Abl, c-Abl) is involved in the regulation of the cell cycle, in the cellular response to genotoxic stress, and in the transmission of information about the cellular environment through integrin signaling. The Abl protein appears to serve a complex role as a cellular module that integrates signals from various extracellular and intracellular sources and that influences decisions in regard to cell cycle and apoptosis. Abelson tyrosine kinase includes sub-type derivatives such as the chimeric fusion (oncoprotein) BCR-Abl with deregulated tyrosine kinase activity or the v-Abl. BCR-Abl is important in the pathogenesis of 95% of chronic myelogenous leukemia (CML) and 10% of acute lymphocytic leukemia. STI-571 (Gleevec) is an inhibitor of the oncogenic BCR-Abl tyrosine kinase and is used for the treatment of chronic myeloid leukemia (CML). However, some patients in the blast crisis stage of CML are resistant to STI-571 due to mutations in the BCR-Abl kinase. Over 22 mutations have been reported to date, such as G250E, E255V, T315I, F317L and M351T.

Compounds of the present invention may inhibit abl kinase, for example, v-abl kinase. The compounds of the present invention may also inhibit wild-type BCR-Abl kinase and mutations of BCR-Abl kinase, and thus may be suitable for the treatment of Bcr-abl-positive cancer and tumor diseases, such as leukemias (especially chronic myeloid leukemia and acute lymphoblastic leukemia, where especially apoptotic mechanisms of action are found). Compounds of the present invention may also be effective against leukemic stem cells, and may be potentially useful for the purification of these cells in vitro after removal of said cells (for example, bone marrow removal), and reimplantation of the cells once they have been cleared of cancer cells (for example, reimplantation of purified bone marrow cells).

PDGF (Platelet-derived Growth Factor) is a commonly occurring growth factor, which plays an important role in normal growth and in pathological cell proliferation, such as in carcinogenesis and in diseases of the smooth-muscle cells of blood vessels, for example in atherosclerosis and thrombosis. Compounds of the invention may inhibit PDGF receptor (PDGFR) activity, and may therefore be suitable for the treatment of tumor diseases, such as gliomas, sarcomas, prostate tumors, and tumors of the colon, breast, and ovary.

Compounds of the present invention may be used not only as a tumor-inhibiting substance, for example in small cell lung cancer, but also as an agent to treat non-malignant proliferative disorders, such as atherosclerosis, thrombosis, psoriasis, scleroderma and fibrosis. Compounds of the present invention may also be useful for the protection of stem cells, for example to combat the hemotoxic effect of chemotherapeutic agents, such as 5-fluoruracil, and in asthma. Compounds of the invention may especially be used for the treatment of diseases which respond to an inhibition of the PDGF receptor kinase.

Compounds of the present invention may exhibit useful effects in the treatment of disorders arising as a result of transplantation, for example, allogenic transplantation, especially tissue rejection, such as obliterative bronchiolitis (OB), i.e. a chronic rejection of allogenic lung transplants. In contrast to patients without OB, those with OB often show an elevated PDGF concentration in bronchoalveolar lavage fluids.

Compounds of the present invention may also be effective against diseases associated with vascular smooth-muscle cell migration and proliferation (where PDGF and PDGF-R often also play a role), such as restenosis and atherosclerosis. These effects and the consequences thereof for the proliferation or migration of vascular smooth-muscle cells in vitro and in vivo may be demonstrated by administration of the compounds of the present invention, and also by investigating its effect on the thickening of the vascular intima following mechanical injury in vivo.

The Tec family kinase, Bmx, a non-receptor protein-tyrosine kinase, controls the proliferation of mammary epithelial cancer cells.

The activity of serum and glucocorticoid-regulated kinase (SGK), is correlated with perturbed ion-channel activities, in particular, those of sodium and/or potassium channels, and compounds of the invention may be useful for treating hypertension.

Certain abnormal proliferative conditions are believed to be associated with raf expression and are therefore believed to be responsive to inhibition of raf expression. Abnormally high levels of expression of the raf protein are also implicated in transformation and abnormal cell proliferation. These abnormal proliferative conditions are also believed to be responsive to inhibition of raf expression. For example, expression of the c-raf protein is believed to play a role in abnormal cell proliferation, since it has been reported that 60% of all lung carcinoma cell lines express unusually high levels of c-raf mRNA and protein. Further examples of abnormal proliferative conditions are hyper-proliferative disorders such as cancers, tumors, hyperplasia, pulmonary fibrosis, angiogenesis, psoriasis, atherosclerosis and smooth muscle cell proliferation in the blood vessels, such as stenosis or restenosis following angioplasty. The cellular signaling pathway of which raf is a part has also been implicated in inflammatory disorders characterized by T-cell proliferation (T-cell activation and growth), such as tissue graft rejection, endotoxin shock, and glomerular nephritis, for example.

The family of human ribosomal S6 protein kinases consists of at least 8 members (RSK1, RSK2, RSK3, RSK4, MSK1, MSK2, p70S6K and p70S6 Kb). Ribosomal protein S6 protein kinases play important pleotropic functions, among them is a key role in the regulation of mRNA translation during protein biosynthesis (Eur. J. Biochem 2000, 267(21): 6321-30; Exp Cell Res. 1999, 253 (1):100-9; Mol Cell Endocrinol. 1999, 151(1-2):65-77). The phosphorylation of the S6 ribosomal protein by p70S6 has also been implicated in the regulation of cell motility (Immunol. Cell Biol. 2000, 78(4):447-51) and cell growth (Prog. Nucleic Acid Res. Mol. Biol. 2000, 65:101-27), and hence, may be important in tumor metastasis, the immune response and tissue repair as well as other disease conditions.

Flt3 (fms-like tyrosine kinase), also known as FLk-2 (fetal liver kinase 2), is a member of the type III receptor tyrosine kinase (RTK) family. Aberrant expression of the Flt3 gene has been documented in both adult and childhood leukemias including acute myeloid leukemia (AML), AML with trilineage myelodysplasia (AML/TMDS), acute lymphoblastic leukemia (ALL), and myelodysplastic syndrome (MDS). Activating mutations of the Flt3 receptor have been found in about 35% of patients with acute myeloblastic leukemia (AML), and are associated with a poor prognosis. The most common mutation involves in-frame duplication within the juxtamembrane domain, with an additional 5-10% of patients having a point mutation at asparagine 835. Both of these mutations are associated with constitutive activation of the tyrosine kinase activity of Flt3, and result in proliferation and viability signals in the absence of ligand. Patients expressing the mutant form of the receptor have been shown to have a decreased chance for cure. Thus, there is accumulating evidence for hyper-activated (mutated) Flt3 kinase activity in human leukemias and myelodysplastic syndrome.

The compounds of the present invention may inhibit cellular processes involving stem-cell factor (SCF, also known as the c-kit ligand or steel factor), such as inhibiting SCF receptor (kit) autophosphorylation and SCF-stimulated activation of MAPK kinase (mitogen-activated protein kinase). MO7e cells are a human promegakaryocytic leukemia cell line, which depend on SCF for proliferation. Compounds of the invention may also inhibit the autophosphorylation of SCF receptors.

Aurora-2 is a serine/threonine protein kinase that has been implicated in human cancer, such as colon, breast and other solid tumors. This kinase is believed to be involved in protein phosphorylation events that regulate the cell cycle. Specifically, Aurora-2 may play a role in controlling the accurate segregation of chromosomes during mitosis. Misregulation of the cell cycle may lead to cellular proliferation and other abnormalities. In human colon cancer tissue, the aurora-2 protein has been found to be overexpressed.

The Aurora family of serine/threonine kinases [Aurora-A (“1”), B (“2”) and C (“3”)] plays an important role in cell proliferation. These proteins are responsible for chromosome segregation, mitotic spindle function and cytokinesis, and are linked to tumorigenesis. Elevated levels of all Aurora family members are observed in a wide variety of tumor cell lines. Aurora kinases are over-expressed in many human tumors reported to be associated with chromosomal instability in mammary tumors. For example, aberrant activity of aurora A kinase has been implicated in colorectal, gastric, human bladder and ovarian cancers. High levels of Aurora-A have also been reported in renal, cervical, neuroblastoma, melanoma, lymphoma, pancreatic and prostate tumor cell lines.

Aurora-B is also highly expressed in multiple human tumor cell lines, for example, leukemic cells and colorectal cancers. Aurora-C, which is normally only found in germ cells, is also over-expressed in a high percentage of primary colorectal cancers and in a variety of tumor cell lines including cervical adenocarcinoma and breast carcinoma cells. Based on the known function of Aurora kinases, inhibition of their activity should disrupt mitosis leading to cell cycle arrest. In vivo, an Aurora inhibitor therefore slows tumor growth and induces regression.

The inactivation of Chk1 and Chk2 abrogates the G2/M arrest which is induced by damaged DNA, and sensitizes the resulting checkpoint deficient cells to the killing by DNA damaging events. As cancer cells are more sensitive towards the abrogation of the G2/M checkpoint than normal cells, there is great interest in compounds which inhibit Chk1, Chk2 or both, abrogate the G2/M checkpoint and improve the killing of cancer cells by DNA damaging events.

It is believed that a wide variety of disease states and conditions may be mediated by modulating the activity of Mammalian Sterile 20-like Kinase, Mst 1 and Mst 2, or combinations thereof, to treat or prevent diseases which include osteoporosis, osteopenia, Paget's disease, vascular restenosis, diabetic retinopathy, macular degeneration, angiogenesis, atherosclerosis, inflammation and tumor growth.

The kinases known as PKA or cyclic AMP-dependent protein kinase, PKB or Akt, and PKC, all play key roles in signal transduction pathways responsible for oncogenesis. Compounds capable of inhibiting the activity of these kinases may be useful in the treatment of diseases characterized by abnormal cellular proliferation, such as cancer.

Rho kinase (Rock-II) participates in vasoconstriction, platelet aggregation, bronchial smooth muscle constriction, vascular smooth muscle proliferation, endothelial proliferation, stress fiber formation, cardiac hypertrophy, Na/H exchange transport system activation, adducing activation, ocular hypertension, erectile dysfunction, premature birth, retinopathy, inflammation, immune diseases, AIDS, fertilization and implantation of fertilized ovum, osteoporosis, brain functional disorder, infection of digestive tracts with bacteria, and the like.

Axl is a receptor tyrosine kinase associated with a number of disease states such as leukemia and various other cancers including gastric cancer.

Bruton's tyrosine kinase (Btk) is important for B lymphocyte development. The Btk family of non-receptor tyrosine kinases includes Btk/Atk, Itk/Emt/Tsk, Bmx/Etk, and Tec. Btk family kinases play central but diverse modulatory roles in various cellular processes. They participate in signal transduction in response to extracellular stimuli resulting in cell growth, differentiation and apoptosis. The aberrant activity of this family of kinases is linked to immunodeficiency diseases and various cancers.

Fibroblast growth factor receptor 3 was shown to exert a negative regulatory effect on bone growth and an inhibition of chondrocyte proliferation. Thanatophoric dysplasia is caused by different mutations in fibroblast growth factor receptor 3, and one mutation, TDII FGFR3, has a constitutive tyrosine kinase activity which activates the transcription factor Stat 1, leading to expression of a cell-cycle inhibitor, growth arrest and abnormal bone development (Su et al., Nature 1997, 386:288-292). FGFR3 is also often expressed in multiple myeloma-type cancers.

An inhibition of tumor growth and vascularization, and also a decrease in lung metastases during adenoviral infections or during injections of the extracellular domain of Tie-2 (Tek) have been shown in breast tumor and melanoma xenograft models (Lin et al., J. Clin. Invest. 1997, 100:2072-2078; Lin et al., Proc Natl. Acad. Sci. 1998, 95:8829-8834). Tie2 inhibitors may be used in situations where neovascularization takes place inappropriately (i.e. in diabetic retinopathy, chronic inflammation, psoriasis, Kaposi's sarcoma, chronic neovascularization due to macular degeneration, rheumatoid arthritis, infantile haemangioma and cancers).

The c-Src kinase transmits oncogenic signals of many receptors. For example, over-expression of EGFR or HER2/neu in tumors leads to the constitutive activation of c-src, which is characteristic of the malignant cell but absent from the normal cell. On the other hand, mice deficient in the expression of c-src exhibit an osteopetrotic phenotype, indicating a key participation of c-src in osteoclast function and a possible involvement in related disorders.

In accordance with the foregoing, the present invention further provides a method for preventing or treating any of the diseases or disorders described above in a subject in need of such treatment, which method comprises administering to said subject a therapeutically effective amount of a compound of Formula (1) or a pharmaceutically acceptable salt thereof. For any of the above uses, the required dosage will vary depending on the mode of administration, the particular condition to be treated and the effect desired. (See, “Administration and Pharmaceutical Compositions,” infra).

Administration and Pharmaceutical Compositions

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention may be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form.

Pharmaceutical compositions comprising a compound of the present invention in free form or in a pharmaceutically acceptable salt form in association with at least one pharmaceutically acceptable carrier or diluent may be manufactured in a conventional manner by mixing, granulating or coating methods. For example, oral compositions may be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets, together with c) binders, e.g., magnesium aluminum silicate, starch paste, gelatin, tragacanth, methylcellulose, sodium carboxymethylcellulose and/or polyvinylpyrrolidone; and if desired, d) disintegrants, e.g., starches, agar, alginic acid or its sodium salt, or effervescent mixtures; and/or e) absorbents, colorants, flavors and sweeteners. Injectable compositions may be aqueous isotonic solutions or suspensions, and suppositories may be prepared from fatty emulsions or suspensions.

The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier may include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, may be aqueous solutions, ointments, creams or gels well-known in the art. Such may contain solubilizers, stabilizers, tonicity enhancing agents, buffers and preservatives.

Compounds of the invention may be administered in therapeutically effective amounts in combination with one or more therapeutic agents (pharmaceutical combinations). For example, synergistic effects may occur with other immunomodulatory or anti-inflammatory substances, for example when used in combination with cyclosporin, rapamycin, or ascomycin, or immunosuppressant analogues thereof, for example cyclosporin A (CsA), cyclosporin G, FK-506, rapamycin, or comparable compounds, corticosteroids, cyclophosphamide, azathioprine, methotrexate, brequinar, leflunomide, mizoribine, mycophenolic acid, mycophenolate mofetil, 15-deoxyspergualin, immunosuppressant antibodies, especially monoclonal antibodies for leukocyte receptors, for example MHC, CD2, CD3, CD4, CD7, CD25, CD28, B7, CD45, CD58 or their ligands, or other immunomodulatory compounds, such as CTLA41g. Where the compounds of the invention are administered in conjunction with other therapies, dosages of the co-administered compounds will vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit may comprise instructions for its administration.

Processes for Making Compounds of the Invention

The present invention also includes processes for the preparation of compounds of the invention. In the reactions described, reactive functional groups desired in the final product (e.g., hydroxy, amino, imino, thio or carboxy groups) may be protected using protecting groups known in the art, to avoid their unwanted participation in the reactions. Conventional protecting groups may be used in accordance with standard practice, for example, see T. W. Greene and P. G. M. Wuts in “Protective Groups in Organic Chemistry”, John Wiley and Sons, 1991.

In one aspect, compounds of Formula (1) wherein =Q-X is a 2-vinyl-1H-pyrrolyl derivative, may be prepared by proceeding as in the following reaction Scheme 1:

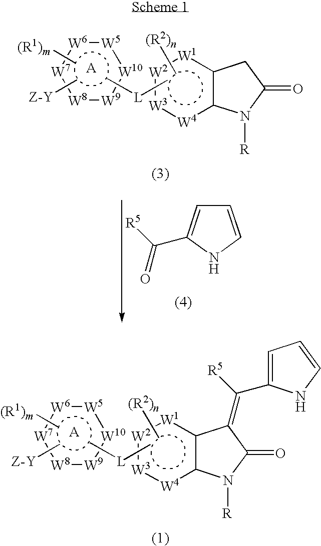

in which W¹, W², W³, W⁴, W⁵, W⁶, W⁷, W⁸, W⁹, W¹⁰, Y, Z, L, R, R¹, R², m and n are as previously defined above;

and R⁵ may be H, alkyl, or any suitable group within the knowledge of those skilled in the art.

As shown in Scheme 1, a compound of Formula (1) may be prepared by reacting a compound of formula (3) with a carbonyl compound (4) in the presence of a suitable base (e.g., piperidine, or the like) and a suitable solvent (e.g., ethanol, or the like). The reaction proceeds in a temperature range of about 50 to about 120° C. and may take up to several hours to complete. In the above scheme 1, pyrrolyl may be further substituted with an optional substituent, such as previously described above.

Detailed examples of the synthesis of a compound of Formula (1) may be found in the Examples, infra.

Additional Processes for Making Compounds of the Invention

A compound of the invention may be prepared as a pharmaceutically acceptable acid addition salt by reacting the free base form of the compound with a pharmaceutically acceptable inorganic or organic acid. Alternatively, a pharmaceutically acceptable base addition salt of a compound of the invention may be prepared by reacting the free acid form of the compound with a pharmaceutically acceptable inorganic or organic base. Alternatively, the salt forms of the compounds of the invention may be prepared using salts of the starting materials or intermediates.

The free acid or free base forms of the compounds of the invention may be prepared from the corresponding base addition salt or acid addition salt form, respectively. For example a compound of the invention in an acid addition salt form may be converted to the corresponding free base by treating with a suitable base (e.g., ammonium hydroxide solution, sodium hydroxide, and the like). A compound of the invention in a base addition salt form may be converted to the corresponding free acid by treating with a suitable acid (e.g., hydrochloric acid, etc.).

Compounds of the invention in unoxidized form may be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention may be prepared by methods known to those of ordinary skill in the art (e.g., for further details see Saulnier et al., Bioorg. Med. Chem. Lett. 1994, 4:1985-90). For example, appropriate prodrugs may be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention may be made by means known to those of ordinary skill in the art. A detailed description of techniques applicable to the creation of protecting groups and their removal may be found in T. W. Greene, “Protecting Groups in Organic Chemistry”, 3^{rd} edition, John Wiley and Sons, Inc., 1999.

Compounds of the present invention may be conveniently prepared or formed during the process of the invention, as solvates (e.g., hydrates). Hydrates of compounds of the present invention may be conveniently prepared by recrystallization from an aqueous/organic solvent mixture, using organic solvents such as dioxin, tetrahydrofuran or methanol.

Compounds of the invention may be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers may be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and may be readily separated by taking advantage of these dissimilarities. The diastereomers may be separated by chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture may be found in Jean Jacques, Andre Collet, Samuel H. Wilen, “Enantiomers, Racemates and Resolutions”, John Wiley And Sons, Inc., 1981.

In summary, the compounds of Formula (1) may be made by a process, which involves:

(a) that of reaction Scheme I;

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

The following examples are offered to illustrate but not to limit the invention. Insofar as the production of the starting materials is not particularly described, the compounds are known or may be prepared analogously to methods known in the art or as disclosed in the Examples hereinafter. One of skill in the art will appreciate that the examples below are only representative of methods for preparation of the compounds of the present invention, and that other well known methods may similarly be used.

### Example 1

### 3-(1H-Pyrrol-2-ylmethylene)-6-{3-[5-(3-trifluoromethoxy-phenyl)-4H-[1,2,4]-triazol-3-yl]-phenylamino}-1,3-dihydro-indol-2-one

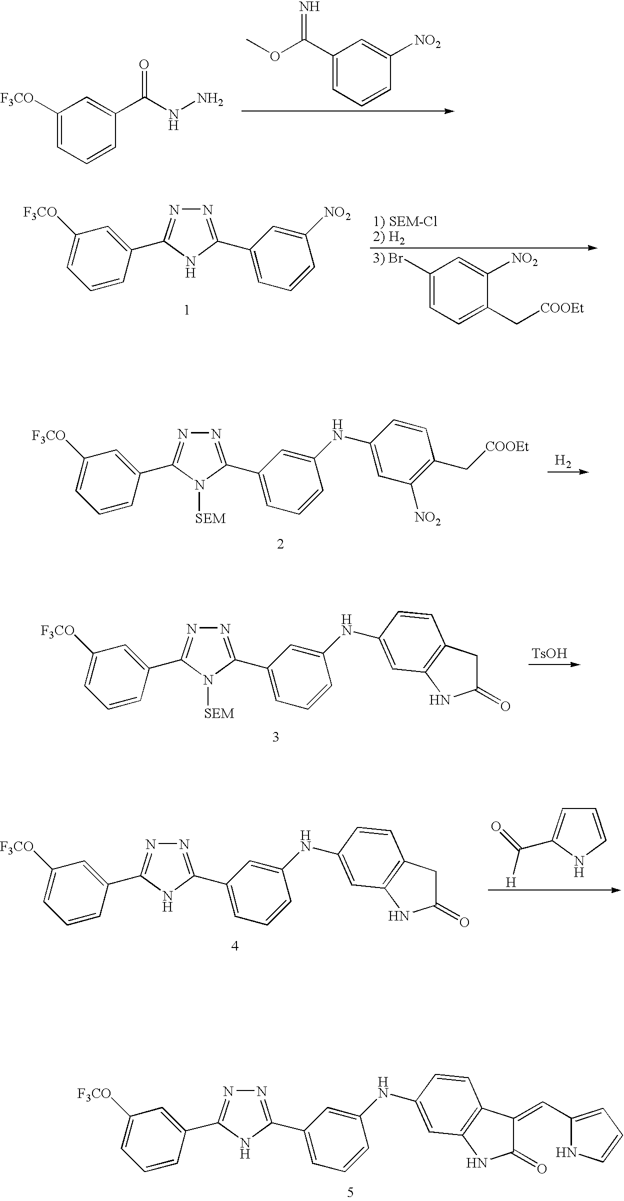

### Synthesis of 3-(3-Nitro-phenyl)-5-(3-trifluoromethoxy-phenyl)-4H-[1,2,4]-triazole (1)

3-Trifluoromethoxybenzoic acid hydrazide (60 mg, 0.27 mmol) and 3-nitro-benzimidic acid methyl ester (0.35 mmol) are mixed in 1,2-dichloroethane (0.5 mL), and is heated at 115° C. for two days. The solvent is evaporated and the residue is purified by column chromatography to give the title compound; LC-MS (m/z) [M⁺+1] 351.2.

### Synthesis of (2-Nitro-4-{3-[5-(3-trifluoromethoxyphenyl)-4-(2-trimethylsilanyl-ethoxymethyl)-4H-[1,2,4]triazol-3-yl]-phenylamino}-phenyl)-acetic acid ethyl ester (2)

To a solution of compound 1 (40 mg, 0.11 mmol) in DMF (2 mL) is added 2-(trimethylsilyl)ethoxymethyl chloride (SEM-Cl, 26 μL) and cesium carbonate (74 mg) subsequently. The suspension is stirred at room temperature for 3 h. The reaction is quenched with water. The mixture is extracted with EtOAc. The combined organic solution is concentrated and purified by column chromatography, which delivers 3-(3-nitro-phenyl)-5-(3-trifluoromethoxyphenyl)-4-(2-trimethylsilanylethoxymethyl)-4H-[1,2,4]triazole. LC-MS (m/z) [M⁺+1] 481.2.

To a solution of 3-(3-nitro-phenyl)-5-(3-trifluoromethoxy-phenyl)-4-(2-trimethylsilanyl-ethoxymethyl)-4H-[1,2,4]-triazole (45 mg, 0.09 mmol) in methanol (4 mL) is added Pa/C (10 wt %, 10 mg). The mixture is stirred under hydrogen balloon overnight. It is filtered through Celite, and concentrated. LC-MS (m/z) [M⁺+1] 451.2.

3-[5-(3-Trifluoromethoxy-phenyl)-4-(2-trimethylsilanyl-ethoxymethyl)-4H-[1,2,4]triazol-3-yl]-phenylamine (38 mg, 0.084 mmol) is mixed with (4-bromo-2-nitro-phenyl)-acetic acid ethyl ester (24 mg), xantaphos (3 mg), palladium(II) acetate (0.8 mg) and cesium carbonate (38 mg) in 1,4-dioxane (1 mL). The mixture is heated at 115° C. for 2 days, then filtered through Celite. The filtrate is concentrated and purified by column chromatography, which yields (2-nitro-4-{3-[5-(3-trifluoromethoxy-phenyl)-4-(2-trimethylsilanyl-ethoxymethyl)-4H-[1,2,4]triazol-3-yl]-phenylamino}-phenyl)-acetic acid ethyl ester. LC-MS (m/z) [M⁺+1] 658.2.

### Synthesis of 6-{3-[5-(3-Trifluoromethoxy-phenyl)-4-(2-trimethylsilanyl-ethoxymethyl)-4H-[1,2,4]triazol-3-yl]-phenylamino}-1,3-dihydro-indol-2-one (3)

To a solution of (2-nitro-4-{3-[5-(3-trifluoromethoxy-phenyl)-4-(2-trimethylsilanyl-ethoxymethyl)-4H-[1,2,4]triazol-3-yl]-phenylamino}-phenyl)-acetic acid ethyl ester (42 mg) in acetic acid (4 mL) is added palladium on carbon (10 wt %, 8 mg). The mixture is stirred at room temperature under hydrogen balloon overnight, and filtered through Celite. The filtrate is concentrated, and the crude is used without further purification. LC-MS (m/z) [M⁺+1] 582.2.

### Synthesis of 6-{3-[5-(3-Trifluoromethoxy-phenyl)-4H-[1,2,4]-triazol-3-yl]-phenylamino}-1,3-dihydro-indol-2-one (4)

To a solution of 6-{3-[5-(3-trifluoromethoxy-phenyl)-4-(2-trimethylsilanyl-ethoxymethyl)-4H-[1,2,4]triazol-3-yl]-phenylamino}-1,3-dihydro-indol-2-one (41 mg) in anhydrous methanol (1 mL) is added p-toluenesulfonic acid monohydrate (15 mg). The mixture is irradiated at 100° C. by microwave for 30 min The mixture is concentrated and purified by column chromatography, which gives the title compound. LC-MS (m/z) [M⁺+1] 452.2.

### Synthesis of 3-(1H-Pyrrol-2-ylmethylene)-6-{3-[5-(3-trifluoromethoxy-phenyl)-4H-[1,2,4]triazol-3-yl]-phenylamino}-1,3-dihydro-indol-2-one (5)

To a suspension of 6-{3-[5-(3-trifluoromethoxy-phenyl)-4H-[1,2,4]triazol-3-yl]-phenylamino}-1,3-dihydro-indol-2-one (14 mg, 0.03 mmol) in EtOH (2 mL) is added pyrrole-2-carboxaldehyde (3.5 mg) and piperidine (12 μL). The mixture is heated at reflux for 2 h, and the solvent is then evaporated. The residue is purified by prep-LC/MS to give the title compound. ¹H NMR (DMSO-d₆) δ 14.66 (s, 1H), 13.17 (s, 1H), 10.78 (s, 1H), 8.64-8.50 (m, 1H), 8.10 (d, 1H), 8.03-7.92 (m, 1H), 7.84 (s, 1H), 7.72-7.40 (m, 6H), 7.30-7.20 (m, 2H), 6.80-6.68 (m, 3H), 6.30 (m, 1H); LC-MS (m/z) [M⁺+1] 529.2.

By repeating the procedures described in the above example, using appropriate starting reagents, the following compounds identified in Table 1 are obtained.

TABLE 1

| Compound | | Physical Data |
|---|---|---|
| No. | Structure | ¹H NMR and/or MS (m/z) |
| 1 | 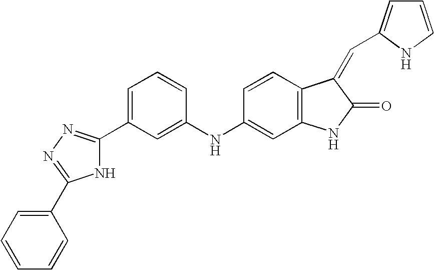 | ¹H NMR (DMSO-d₆) δ 14.51 (s, 1 H), 13.19 (s, 1 H), 10.80 (s, 1 H), 8.57 (d, 1 H), 8.08 (dd, 2 H), 7.94 (d, 1 H), 7.60-7.35 (m, 7 H), 7.28 (s, 1 H), 7.20 (dd, 1 H), 6.82-6.70 (m, 3 H), 6.35-6.25 (m, 1 H); LC- MS (m/z) [M⁺ + 1] 445.2. |
| 2 | 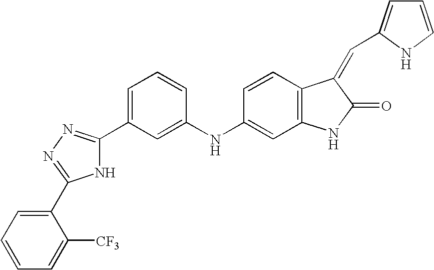 | ¹H NMR (DMSO-d₆) δ 13.17 (s, 1 H), 10.78 (s, 1 H), 8.78 (br s, 1 H), 7.95-7.75 (m, 4 H), 7.72 (t, 1 H), 7.55-7.45 (m, 3 H), 7.41 (t, 1 H), 7.30-7.25 (m, 1 H), 7.25-7.15 (m, 1 H), 6.77 (dd, 1 H), 6.75-6.70 (m, 1 H), 6.70 (d, 1 H), 6.35-6.30 (m, 1 H); LC-MS (m/z) [M⁺ + 1] 513.2. |
| 3 | 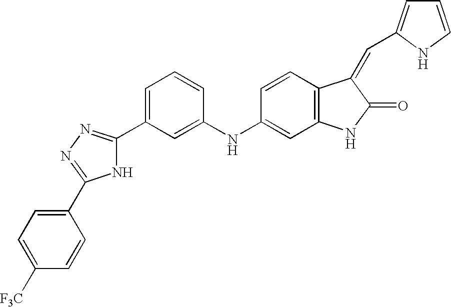 | ¹H NMR (DMSO-d₆) δ 14.70 (br s, 1 H), 13.17 (s, 1 H), 10.79 (s, 1 H), 8.60 (br s, 1 H), 8.29(s, 1 H), 8.26 (s, 1 H), 7.95-7.80 (m, 3 H), 7.60-7.40 (m, 4 H), 7.30-7.20 (m, 2 H), 6.80-6.65 (m, 3 H), 6.35- 6.30 (m, 1 H); LC-MS (m/z) [M⁺ + 1] 513.2 |
| 4 | 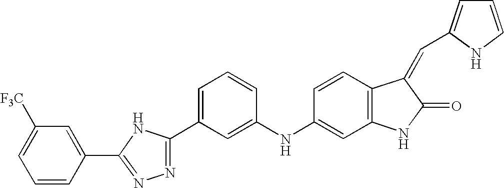 | LC-MS (m/z) [M⁺ + 1] 513.2. |
| 5 | 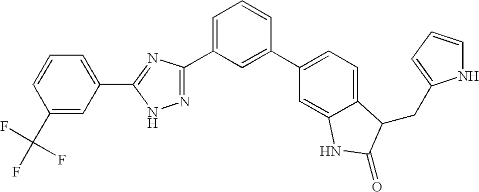 | ¹H NMR (DMSO-d₆) δ 6.36-6.40 (m, 1H), 6.88 (m, 1H), 7..24 (s, 1H), 7.39 (3, 1H), 7.40-7.48 (m, 1H), 7.60-7.70 (m, 1H), 7.72-7.90 (m, 5H), 8.09 (d, 1H), 8.38 (s, 2H), 8.41 (d, 1H), 11.09 (s, 1H), 13.34 (s, 1H), 14.83 (s, 1H); LC-MS (m/z) [M⁺ + 1] 498.2. |
| 6 | 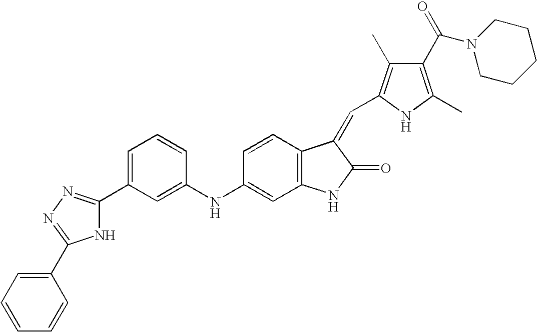 | ¹H NMR (DMSO-d₆) δ 13.36 (s, 1 H), 10.80 (s, 1 H), 8.15-8.00 (m, 2 H), 7.90-7.85 (m, 1 H), 7.70-7.60 (m, 1 H), 7.60-7.35 (m, 7 H), 7.22- 7.15 (m, 1 H), 6.80-6.70 (m, 2 H), 3.60-3.20 (m, 4 H), 2.67 (s, 3 H), 2.22 (s, 3 H), 1.70-1.40 (m, 6 H); LC-MS (m/z) [M⁺ + 1] 584.2. |
| 7 | 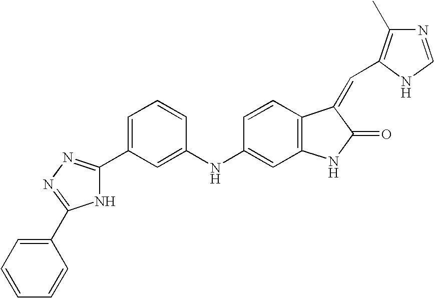 | ¹H NMR (DMSO-d₆) δ 13.16 (s, 1 H), 14.90 (br s, 1 H), 11.27 (s, 1 H), 9.02 (d, 1 H), 8.90 (br s, 1 H), 8.10 (s, 1 H), 8.08 (s, 1 H), 7.92 (s, 1 H), 7.79 (d, 1 H), 7.65 (d, 1 H), 7.60 (s, 1 H), 7.55-7.40 (m, 4 H), 7.25 (dd, 1 H), 6.82 (dd, 1 H), 6.74 (d, 1 H), 2.58 (s, 1 H); LC-MS (m/z) [M⁺ + 1] 460.2. |
| 8 | 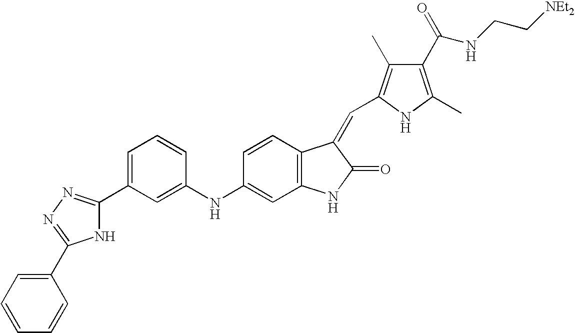 | ¹H NMR (DMSO-d₆) δ 14.50 (br s, 1 H), 13.53 (s, 1 H), 10.83 (s, 1 H), 9.24 (br s, 1 H), 8.56 (br s, 1 H), 8.07 (d, 1 H), 8.06 (s, 1 H), 7.86 (s, 1 H), 7.74 (t, 1 H), 7.67 (d, 1 H), 7.60-7.35 (m, 5 H), 7.18 (d, 1 H), 6.78 (dd, 1 H), 6.72 (d, 1 H), 3.60-3.50 (m, 1 H), 3.30-3.15 (m, 6 H), 2.46 (s, 3 H), 2.41 (s, 3 H), 1.23 (t, 6 H); LC-MS (m/z) [M⁺ + 1] 614.2. |
| 9 | 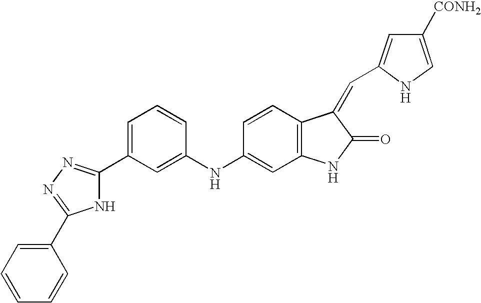 | ¹H NMR (DMSO-d₆) δ 13.32 (s, 1 H), 10.89 (s, 1 H), 8.10 (s, 1 H), 8.08 (s, 1 H), 7.88 (s, 1 H), 7.72 (s, 1H), 7.65-7.35 (m, 9 H), 7.22 (d, 1 H), 7.07 (s, 1 H), 7.78 (d, 1 H), 6.73 (s, 1 H); LC-MS (m/z) [M⁺ + 1] 488.2. |
| 10 | 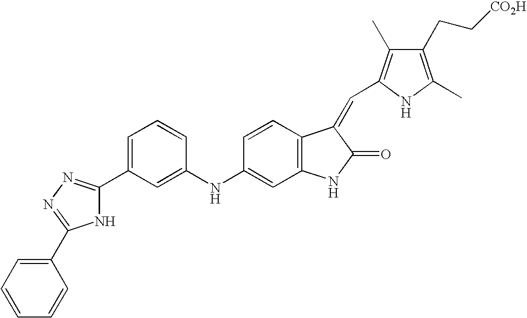 | ¹H NMR (mixture of two geometric isomers, DMSO-d₆) δ 13.57 (s, 0.7 H), 13.17 (s, 0.3 H), 10.97 (s, 0.7 H), 10.67 (s, 0.3 H), 9.75 (s, 1 H), 8.10-8.00 (m, 2 H), 7.90-7.80 (m, 1 H), 7.80-7.70 (m, 1 H), 7.65-7.30 (m, 7 H), 7.25- 7.15 (m, 1 H), 6.80-6.65 (m, 2 H), 2.98 (t, 1.4 H), 2.62 (t, 0.6 H), 2.34 (t, 1.4 H), 2.30-2.15 (m, 6 H + 0.6 H); LC-MS (m/z) [M⁺ + 1] 545.2. |
| 11 | 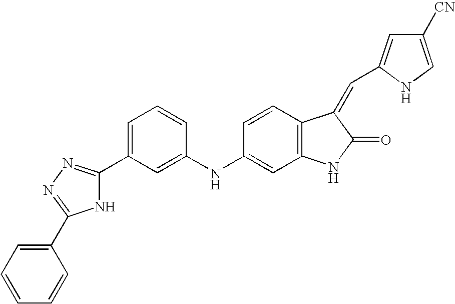 | ¹H NMR (DMSO-d₆) δ 13.67 (s, 1 H), 11.00 (s, 1 H), 8.70 (br s, 1 H), 8.10 (s, 1 H), 8. 08 (s, 1 H), 7.95 (s, 1 H), 7.89 (s, 1 H), 7.61 (d, 1 H), 7.55-7.45 (m, 6 H), 7.43 (t, 1 H), 7.23 (d, 1 H), 7.10 (s, 1 H), 6.79 (d, 1 H), 6.73 (s, 1 H); LC- MS (m/z) [M⁺ + 1] 470.2. |
| 12 | 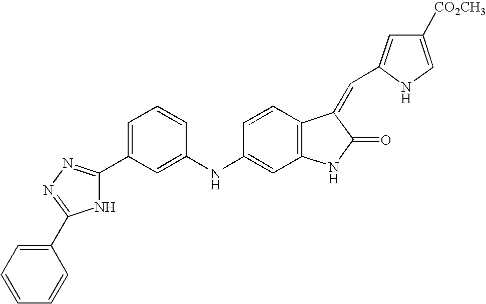 | ¹H NMR (DMSO-d₆) δ 10.91 (s, 1 H), 8.66 (br s , 1 H), 8.08 (s, 1 H), 8.06 (s, 1 H), 7.87 (s, 1 H), 7.81 (s, 1 H), 7.60-7.40 (m, 8 H), 7.25- 7.15 (m, 1 H), 7.06 (s, 1 H), 6.78 (dd, 1 H), 6.71 (d, 1 H), 3.75 (s, 1 H); LC-MS (m/z) [M⁺ + 1] 503.2. |
| 13 | 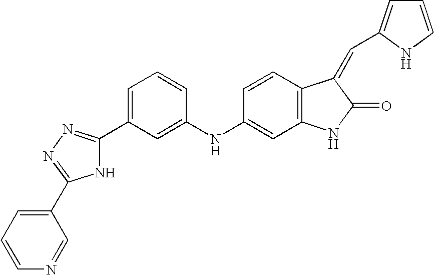 | ¹H NMR (DMSO-d₆) δ 13.17 (s, 1 H), 10.79 (s, 1 H), 9.24 (d, 1 H), 8.65 (d, 1 H), 8.58 (s, 1 H), 8.38 (dt, 1 H), 7.86 (dd, 1 H), 7.60-7.45 (m, 5 H), 7.42 (t, 1 H), 7.30-7.25 (m, 1 H), 7.25-7.15 (m, 1 H), 6.80- 6.65 (m, 3 H), 6.35-6.25 (m, 1 H); LC-MS (m/z) [M⁺ + 1] 446.2. |
| 14 | 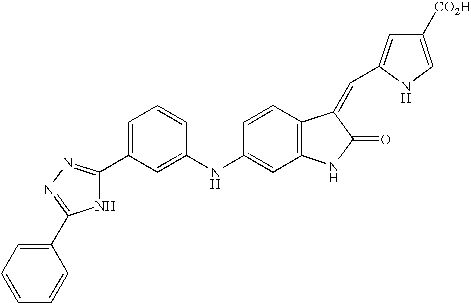 | ¹H NMR (DMSO-d₆) δ 13.38 (s, 1 H), 10.90 (s, 1 H), 8.65 (br s, 1 H), 8.08 (s, 1 H), 8.06 (s, 1 H), 7.87 (s, 1 H), 7.75 (d, 1 H), 7.60-7.45 (m, 7 H), 7.41 (t, 1 H), 7.21 (dd, 1 H), 7.02 (s, 1 H), 6.77 (d, 1 H), 6.71 (d, 1 H); LC-MS (m/z) [M⁺ + 1] 489.2. |
| 15 | 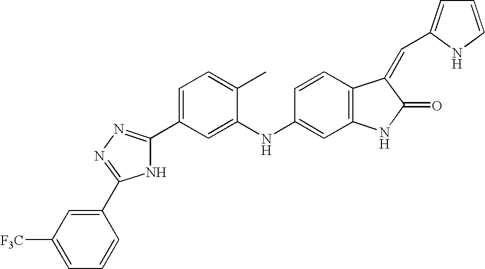 | ¹H NMR (DMSO-d₆) δ 14.62 (br s, 1 H), 13.16 (s, 1 H), 10.74 (s, 1 H), 8.35-8.25 (m, 2 H), 8.04 (d, 1 H), 7.85-7.60 (m, 3 H), 7.66 (dd, 1 H), 7.50-7.40 (m, 2 H), 7.45 (d, 1 H), 7.30-7.20 (m, 1 H), 6.75-6.65 (m, 1 H), 6.68 (dd, 1 H), 6.60 (d, 1 H), 6.35-6.25 (m, 1 H), 2.28 (s, 3 H). |
| 16 | 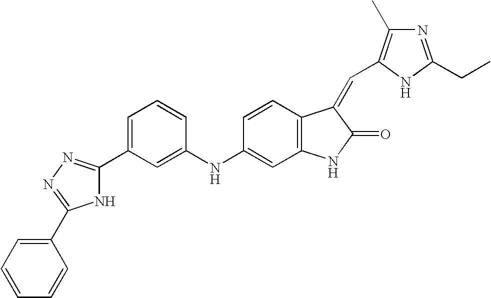 | ¹H NMR (mixture of two geometric isomers, DMSO-d₆) δ 14.51 (s, 1 H), 13.64 (s, 1 H), 10.83 (s, 1 H), 8.59 (s, 1 H), 8.08 (s, 1 H), 8.06 (s, 1 H), 7.88 (s, 1 H), 7.67 (d, 1 H), 7.60-7.35 (m, 6 H), 7.25-7.15 (m, 1 H), 6.77 (dd, 1 H), 6.72 (d, 1 H), 2.74 (q, minor isomer), 2.60 (q, major isomer), 2.38 (s, 3 H), 1.26 (t, minor isomer), 1.19 (t, major isomer); LC-MS (m/z) [M⁺ + 1] 488.2. |
| 17 | 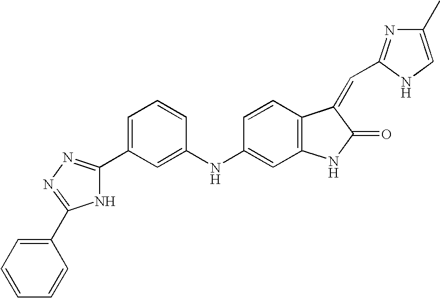 | ¹H NMR (DMSO-d₆) δ 14.52 (s, 1 H), 10.98 (s, 1 H), 8.80-8.65 (m, 1 H), 8.08 (s, 1 H), 8.06 (s, 1 H), 1.89 (s, 1 H), 7.70-7.35 (m, 6 H), 7.30-7.15 (m, 2 H), 7.04 (s, 1 H), 7.76 (d, 1 H), 7.71 (d, 1 H), 2.24 (s, 3 H); LC-MS (m/z) [M⁺ + 1] 460.2. |
| 18 | 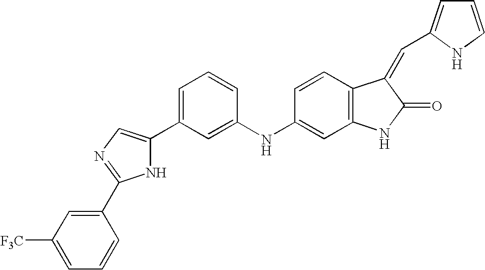 | ¹H NMR (DMSO-d₆) δ 13.16 (s, 1 H), 12.95 (s, 1 H), 10.75 (s, 1 H), 8.43 (s, 1 H), 8.34 (s, 1 H), 8.30- 8.25 (m, 1 H), 7.82 (s, 1 H), 7.72 (s, 1 H), 77.1 (s, 1 H), 7.67 (s, 1 H), 7.40-7.20 (m, 3 H), 7.05-6.95 (m, 1 H), 6.75-6.65 (m, 3 H), 6.35-6.30 (m, 1 H); LC-MS (m/z) [M⁺ + 1] 512.2. |
| 19 | 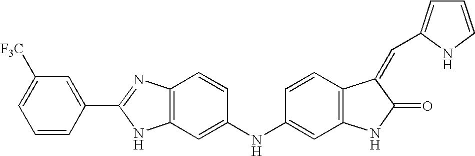 | ¹H NMR (DMSO-d₆) δ 13.17 (s, 1 H), 10.84 (s, 1 H), 8.70 (br s, 1 H), 8.50 (d, 1 H), 8.03 (d, 1 H), 7.93 (t, 1 H), 7.72 (d, 1 H), 7.55-7.50 (m, 2 H), 7.40 (s, 1 H), 7.30-7.20 (m, 2 H), 6.80 (d, 1 H), 6.75-6.70 (m, 2 H), 6.35-6.30 (m, 1 H); LC- MS (m/z) [M⁺ + 1] 486.2. |
| 20 | 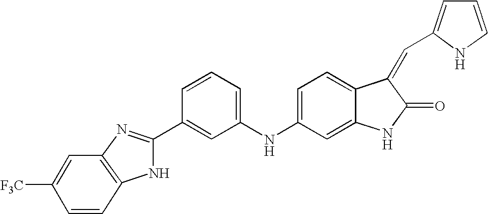 | ¹H NMR (DMSO-d₆) δ 13.19 (s, 1 H), 10.82 (s, 1 H), 8.70 (brs, 1 H), 8.05-7.95 (m, 2 H), 7.87 (d, 1 H), 7.68 (dd, 2 H), 7.60-7.45 (m, 3 H), 7.30-7.25 (m, 2 H), 6.83 (dd, 1 H), 6.80-6.70 (m, 2 H), 6.3 5-6.30 (m, 1 H); LC-MS (m/z) [M⁺ + 1] 486.2. |
| 21 | 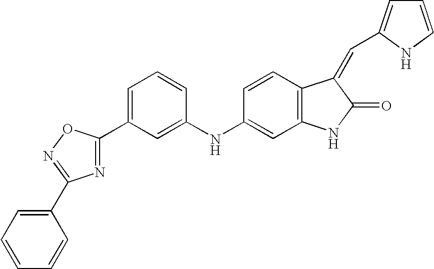 | ¹H NMR (DMSO-d₆) δ 13.18 (s, 1 H), 10.81 (s, 1 H), 8.67 (s, 1 H), 8.29 (dd, 2 H), 7.85-7.80 (m, 1 H), 7.75-7.70 (m, 1 H), 7.70-7.65 (m, 2 H), 7.60-7.50 (m, 3 H), 7.46 (t, 1 H), 7.35-7.30 (m, 1 H), 7.30-7.25 (m, 1 H), 7.79 (dd, 1 H), 6.75-6.70 (m, 2 H), 6.35-6.30 (m, 1 H); LC- MS (m/z) [M⁺ + 1] 446.2. |
| 22 | 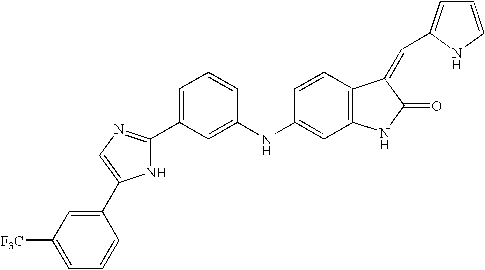 | ¹H NMR (DMSO-d₆) δ 6.30-6.33 (m, 1H), 6.70 (d, 1H), 6.72-6.75 (m, 1H), 6.76-6.84 (dd, 1H), 7.16 (d, 1H), 7.28 (s, 1H), 7.40-7.54 (m, 4H), 7.62-7.74 (m, 2H), 7.81 (s, 1H), 8.18 (d, 2H), 8.25 (s, 1H), 8.61 (s, 1H), 10.80 (s, 1H), 13.17 (s, 1H); LC-MS (m/z) [M⁺ + 1] 512.2. |
| 23 | 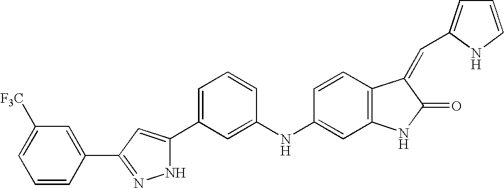 | LC-MS (m/z) [M⁺ + 1] 512.2. |
| 24 | 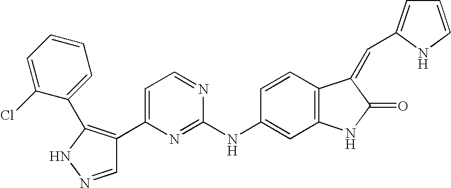 | ¹H NMR (DMSO-d₆) δ 6.30 (d, 1H), 6.34-6.38 (m, 1H), 6.73 (d, 1H), 6.85-6.90 (m, 1H), 7.22-7.30 (dd, 1H), 7.36 (s, 1H), 7.40-7.60 (m, 5H), 7.85 (d, 1H), 8.21 (d, 1H), 8.34 (s, 1H), 9.35 (s, 1H), 10.80 (s, 1H), 13,39 (s, 1H); LC- MS (m/z) [M⁺ + 1] 480.1. |
| 25 | 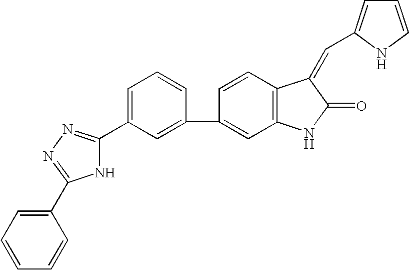 | |
| 26 | 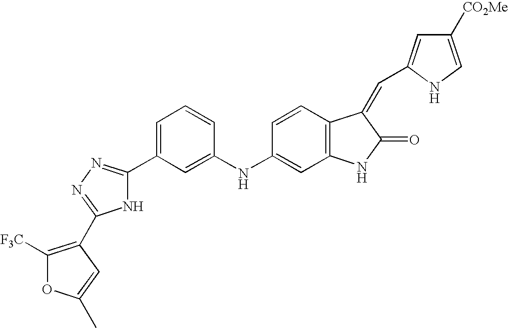 | |
| 27 | 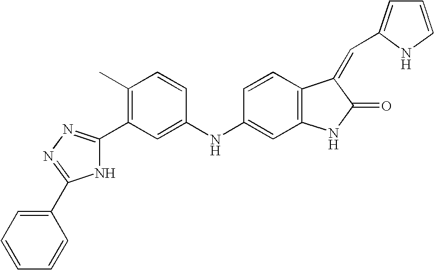 | |
| 28 | 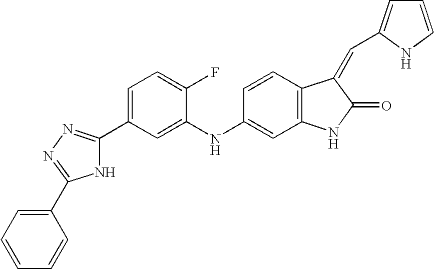 | |
| 29 | 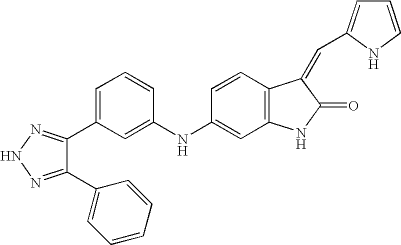 | |
| 30 | 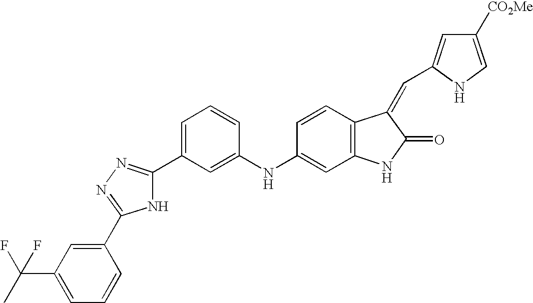 | |
| 31 | 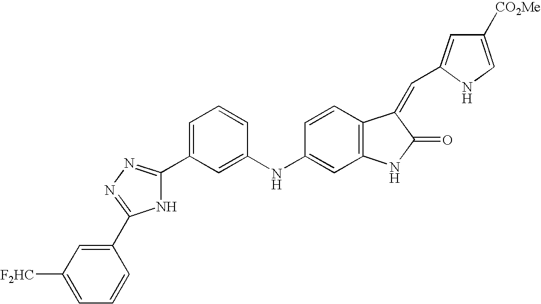 | |
| 32 | 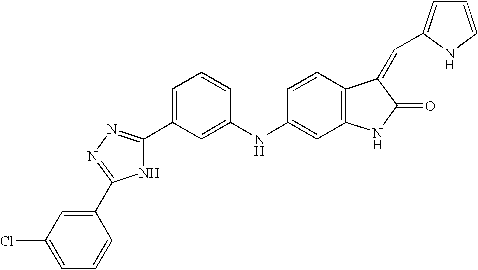 | |
| 33 | 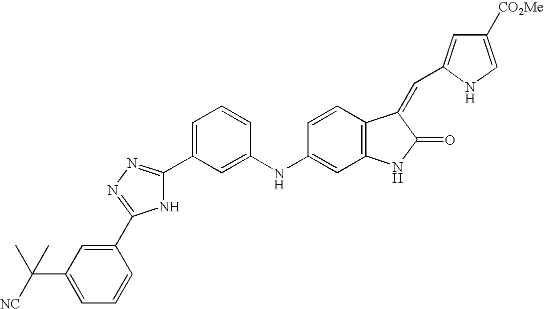 | |

### Assays

Compounds of the present invention may be assayed to measure their capacity to selectively inhibit cell proliferation of 32D cells expressing BCR-Abl (32D-p210) compared with parental 32D cells. Compounds selectively inhibiting the proliferation of these BCR-Abl transformed cells are tested for anti-proliferative activity on Ba/F3 cells expressing either wild type or the mutant forms of Bcr-abl.

Compounds of the present invention may also be assayed to measure their capacity to selectively inhibit cell proliferation of Ba/F3 cells expressing ETV6-NTRK3 (Ba/F3 EN) compared with parental Ba/F3 cells. Compounds selectively inhibiting the proliferation of these ETV6-NTRK3 transformed cells are tested for anti-proliferative activity on Ba/F3 cells expressing either Tel fusions of Trk family members, specifically NTRK1 and NTRK2. In addition, compounds may be assayed to measure their capacity to inhibit TrkA, TrkB, TrkC, Abl, Bcr-Abl, cSrc, TPR-Met, Tie2, MET, FGFR3, Aurora, Axl, Bmx, BTK, c-kit, CHK2, Flt3, MST2, p70S6K, PDGFR, PKB, PKCα, Raf, ROCK-II, Rsk1, and SGK kinases.

Inhibition of Cellular BCR-Abl Dependent Proliferation (High Throughput Method)

The murine cell line 32D hemopoietic progenitor cell line may be transformed with BCR-Abl cDNA (32D-p210). These cells are maintained in RPMI/10% fetal calf serum (RPMI/FCS) supplemented with penicillin 50 μg/mL, streptomycin 50 μg/mL and L-glutamine 200 mM. Untransformed 32D cells are similarly maintained with the addition of 15% of WEHI conditioned medium as a source of IL3.

50 μl of a 32D or 32D-p210 cells suspension are plated in Greiner 384 well microplates (black) at a density of 5000 cells per well. 50 mL of test compound (1 mM in DMSO stock solution) is added to each well (STI571 is included as a positive control). The cells are incubated for 72 hours at 37° C., 5% CO₂. 10 μl of a 60% Alamar Blue solution (Tek diagnostics) is added to each well and the cells are incubated for an additional 24 hours. The fluorescence intensity (excitation at 530 nm; emission at 580 nm) is quantified using the Acquest™ system (Molecular Devices).

Inhibition of Cellular BCR-Abl Dependent Proliferation

32D-p210 cells are plated into 96 well TC plates at a density of 15,000 cells per well. 50 μl of two fold serial dilutions of the test compound (Cₘₐₓ is 40 μM) are added to each well (STI571 is included as a positive control). After incubating the cells for 48 hours at 37° C., 5% CO₂, 15 μL of MTT (Promega) is added to each well and the cells are incubated for an additional 5 hours. The optical density at 570 nm is quantified spectrophotometrically and IC₅₀ values, the concentration of compound required for 50% inhibition, are determined from a dose response curve.

Effect on Cell Cycle Distribution

32D and 32D-p210 cells are plated into 6 well TC plates at 2.5×10⁶ cells per well in 5 ml of medium, and test compound at 1 or 10 μM is added (STI571 is included as a control). The cells are then incubated for 24 or 48 hours at 37° C., 5% CO₂. A two ml of cell suspension is washed with PBS, fixed in 70% EtOH for 1 hour and treated with PBS/EDTA/RNase A for 30 minutes. Propidium iodide (Cf=10 μg/ml) is added and the fluorescence intensity is quantified by flow cytometry on the FACScalibur™ system (BD Biosciences). In some embodiments, test compounds of the present invention may demonstrate an apoptotic effect on the 32D-p210 cells but not induce apoptosis in the 32D parental cells.

Effect on Cellular Bcr-Abl Autophosphorylation

BCR-Abl autophosphorylation is quantified with capture Elisa using a c-abl specific capture antibody and an antiphosphotyrosine antibody. 32D-p210 cells are plated in 96 well TC plates at 2×10⁵ cells per well in 50 μL of medium. 50 zμL of two fold serial dilutions of test compounds (Cₘₐₓ is 10 μM) are added to each well (STI571 is included as a positive control). The cells are incubated for 90 minutes at 37° C., 5% CO₂. The cells are then treated for 1 hour on ice with 150 μL of lysis buffer (50 mM Tris-HCl, pH 7.4, 150 mM NaCl, 5 mM EDTA, 1 mM EGTA and 1% NP-40) containing protease and phosphatase inhibitors. 50 μL of cell lysate is added to 96 well optiplates previously coated with anti-abl specific antibody and blocked. The plates are incubated for 4 hours at 4° C. After washing with TBS-Tween 20 buffer, 50 μL of alkaline-phosphatase conjugated anti-phosphotyrosine antibody is added, and the plate is further incubated overnight at 4° C. After washing with TBS-Tween 20 buffer, 90 μL of a luminescent substrate is added and the luminescence is quantified using the Acquest™ system (Molecular Devices). In some embodiments, test compounds of the invention may inhibit the proliferation of the BCR-Abl expressing cells, inhibiting the cellular BCR-Abl autophosphorylation in a dose-dependent manner.

Effect on Proliferation of Cells Expressing Mutant Forms of Bcr-Abl

Compounds of the invention may be tested for their antiproliferative effect on Ba/F3 cells expressing either wild type or the mutant forms of BCR-Abl (G250E, E255V, T315I, F317L, M351T) that confers resistance or diminished sensitivity to STI571. The antiproliferative effect of these compounds on the mutant-BCR-Abl expressing cells and on the non transformed cells may be tested at 10, 3.3, 1.1 and 0.37 μM as described above (in media lacking IL3). The IC₅₀ values of the compounds lacking toxicity on the untransformed cells are determined from the dose response curves obtained as described above.

FLT3 and PDGFRβ

The effects of compounds of the invention on the cellular activity of FLT3 and PDGFRβ may be conducted using the following method. For FLT3 and PDGFRβ, Ba/F3-FLT3-ITD and Ba/F3-Tel-PDGFRβ are used, respectively.

Compounds of the invention may be tested for their ability to inhibit transformed Ba/F3-FLT3-ITD or Ba/F3-Tel-PDGFRβ cells proliferation, which is dependent on FLT3 or PDGFRβ cellular kinase activity. Ba/F3-FLT3-ITD or Ba/F3-Tel-PDGFRβ are cultured up to 800,000 cells/mL in suspension, with RPMI 1640 supplemented with 10% fetal bovine serum as the culture medium. Cells are dispensed into 384-well format plate at 5000 cell/well in 50 μL culture medium. Compounds of the invention are dissolved and diluted in dimethylsulfoxide (DMSO). Twelve points 1:3 serial dilutions are made into DMSO to create concentrations gradient ranging typically from 10 mM to 0.05 μM. Cells are added with 50 nL of diluted compounds and incubated for 48 hours in cell culture incubator. AlamarBlue® (TREK Diagnostic Systems), which may be used to monitor the reducing environment created by proliferating cells, are added to cells at a final concentration of 10%. After additional four hours of incubation in a 37° C. cell culture incubator, fluorescence signals from reduced AlamarBlue® (excitation at 530 nm; emission at 580 nm) are quantified on Analyst GT (Molecular Devices Corp.). IC₅₀ values are calculated by linear regression analysis of the percentage inhibition of each compound at 12 concentrations.

Inhibition of Cellular ETV6-NTRK3 Dependent Proliferation (High Throughput Method)

The murine cell line Ba/F3 hematopoietic progenitor cell line may be transformed with ETV6-NTRK3 cDNA (Ba/F3 EN). These cells are maintained in RPMI/10% fetal calf serum (RPMI/FCS) supplemented with penicillin 50 μg/mL, streptomycin 50 μg/mL and L-glutamine 200 mM. Untransformed Ba/F3 cells are similarly maintained with the addition of 10% of WEHI conditioned medium as a source of IL3.

50 μl of a Ba/F3 or Ba/F3 EN cells suspension are plated in Greiner 384 well microplates (black) at a density of 2000 cells per well. 50 nL of test compound (1 mM in DMSO stock solution) is added to each well. The cells are incubated for 72 hours at 37° C., 5% CO₂. 10 μl of a 60% Alamar Blue solution (Tek diagnostics) is added to each well and the cells are incubated for an additional 24 hours. The fluorescence intensity (excitation at 530 nm; emission at 580 nm) is quantified using the Acquest™ system (Molecular Devices).

Inhibition of Cellular ETV6-NTRK3 Dependent Proliferation

10,000 cells per well contained in 90 μL of media Ba/F3 EN cells are plated into 96 well TC plates. 10 μL of three fold serial dilutions of the test compound (Cₘₐₓ is 10 μM) are added to each well (STI571 is included as a positive control). After incubating the cells for 72 hours at 37° C., 5% CO₂, 15 μL of MTT (Promega) is added to each well and the cells are incubated for an additional 5 hours. The optical density at 570 nm is quantified spectrophotometrically and IC₅₀ values are determined from a dose response curve.

Effect on Proliferation of Cells

Compounds of the invention may be tested for their antiproliferative effect on Ba/F3 cells expressing either ETV6-NTRK3 or ETV6-NTRK1, ETV6-NTRK2, Bcr-Abl, FLT3, FGFR3, NPM-Alk, FIG-Ros and Ror1. The antiproliferative effect of these compounds on the different cell lines and on the non transformed cells are tested at 3-fold serial dilutions in 384 well plates as described above (in media lacking IL3). The IC₅₀ values of the compounds in different cell lines are determined from the dose response curves obtained as described above.

Upstate KinaseProfiler™—Radio-Enzymatic Filter Binding Assay

Compounds of the invention may be assessed for their ability to inhibit individual members of a panel of kinases (a partial, non-limiting list of kinases includes: TrkA, TrkB, TrkC, Abl, Bcr-Abl, cSrc, TPR-Met, Tie2, MET, FGFR3, Aurora, Axl, Bmx, BTK, c-kit, CHK2, Flt3, MST2, p70S6K, PDGFR, PKB, PKCα, Raf, ROCK-II, Rsk1, and SGK kinases.

The compounds are tested in duplicates at a final concentration of 10 μM following this generic protocol. Note that the kinase buffer composition and the substrates vary for the different kinases included in the Upstate Kinase Profiler™ panel. Kinase buffer (2.5 μL, 10×-containing MnCl₂ when required), active kinase (0.001-0.01 Units; 2.5 μL), specific or Poly(Glu-4-Tyr) peptide (5-500 μM or 0.01 mg/ml) in kinase buffer and kinase buffer (50 μM; 5 μL) are mixed in an eppendorf on ice. A Mg/ATP mix (10 μL; 67.5 (or 33.75) mM MgCl₂, 450 (or 225) μM ATP and 1 μCi/μl [γ-³²P]-ATP (3000 Ci/mmol)) is added and the reaction is incubated at about 30° C. for about 10 minutes. The reaction mixture is spotted (20 μL) onto a 2 cm×2 cm P81 (phosphocellulose, for positively charged peptide substrates) or Whatman No. 1 (for Poly (Glu-4-Tyr) peptide substrate) paper square. The assay squares are washed four times for five minutes each with 0.75% phosphoric acid, and washed once with acetone for 5 minutes. The assay squares are transferred to a scintillation vial, 5 ml scintillation cocktail is added and ³²P incorporation (cpm) to the peptide substrate is quantified with a Beckman scintillation counter. Percentage inhibition is calculated for each reaction.

Compounds of Formula (1) or (2) in free form or in pharmaceutically acceptable salt form may exhibit valuable pharmacological properties, for example, as indicated by the in vitro tests described in this application. The IC₅₀ value in those experiments is given as that concentration of the test compound in question that results in a cell count that is 50% lower than that obtained using the control without inhibitor. In general, compounds of the invention have IC₅₀ values from 1 nM to 10 μM. In some examples, compounds of the invention have IC₅₀ values from 0.01 μM to 5 μM. In other examples, compounds of the invention have IC₅₀ values from 0.01 μM to 1 μM, or more particularly from 1 nM to 1 μM. In yet other examples, compounds of the invention have IC₅₀ values of less than 1 nM or more than 10 μM. Compounds of Formula (1) or (2) may exhibit a percentage inhibition of greater than 50%, or in other embodiments, may exhibit a percentage inhibition greater than about 70%, against one or more of the following kinases at 10 μM: TrkA, TrkB, TrkC, Abl, Bcr-Abl, cSrc, TPR-Met, Tie2, MET, FGFR3, Aurora, Axl, Bmx, BTK, c-kit, CHK2, Flt3, MST2, p70S6K, PDGFR, PKB, PKCα, Raf, ROCK-II, Rsk1, and SGK kinases.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A compound having Formula (1): 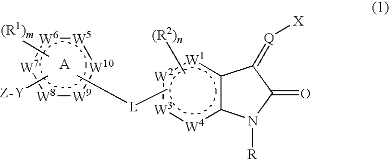
or pharmaceutically acceptable salts and tautomers thereof, wherein:
W¹, W², W³, W⁴, W⁵, W⁶, W⁷, W⁸, W⁹ and W¹⁰ are independently C or N; provided each of W¹, W², W³, W⁴, W⁵, W⁶, W⁷, W⁸, W⁹ and W¹⁰ is C when attached to L, Y, R¹ and R²;
Q is N, NNR, NO or CR⁰;
L is a bond, —O—, —NRC(O)—, —NRC(O)NR—, —C(O)NR, —NR— or S;
R⁰, R¹ and R² are independently halo; C₁₋₆alkyl, C₂₋₆ alkenyl, or C₃₋₆ alkynyl, each of which may be optionally halogenated or optionally substituted with N, O or S; or an optionally substituted aryl, heteroaryl, carbocyclic ring or heterocyclic ring; or R⁰ is H;
each R is H or C₁₋₆alkyl;
X and Z are independently an optionally substituted aryl, heteroaryl, heterocyclic ring or carbocyclic ring;
Y is an optionally substituted heteroaryl;
alternatively, Ring A together with Y may form a fused heteroaryl; or Y and Z together may form a fused heteroaryl;
m is 0-4; and
n is 0-3;
provided said compound is not 3-(1H-pyrrol-2-ylmethylene)-6-{3-[3-(3-trifluoromethyl-phenyl)-[1,2,4]oxadiazol-5-yl]-phenylamino}-1,3-dihydro-indol-2-one.

2. The compound of claim 1, wherein X, Y and Z are independently an optionally substituted 5-7 membered heteroaryl having N, O or S; or Z is an optionally substituted 5-7 membered aryl.

3. The compound of claim 1, wherein X and Y are independently an optionally substituted pyrrolyl, imidazolyl, triazolyl, tetrazolyl, pyridyl, pyrimidinyl, oxazolyl, isoxazolyl, pyrazolyl, or furanyl; or Ring A together with Y form benzimidazolyl.

4. The compound of claim 1, wherein Z is an optionally substituted phenyl, pyridyl or furanyl; or Y and Z together form benzimidazolyl.

5. The compound of claim 1, wherein R¹ and R² are independently halo, or an optionally halogenated C₁₋₆ alkyl or C₁₋₆ alkoxy.

6. The compound of claim 1, wherein L is a bond or NH.

7. The compound of claim 1, wherein Q is CR⁰ and R⁰ is H or C₁₋₆alkyl.

8. The compound of claim 1, wherein each W¹, W², W³, W⁴, W⁵, W⁶, W⁷, W⁸, W⁹ and W¹⁰ is C.

9. The compound of claim 1, wherein two of W⁵, W⁶, W⁷, W⁸, W⁹ and W¹⁰ are N and the others are C.

10. The compound of claim 1, wherein said compound is of Formula (2): 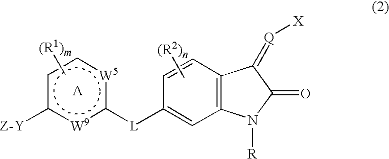
wherein R¹ and R² are independently halo, or an optionally halogenated C₁₋₆ alkyl or C₁₋₆ alkoxy;
W⁵ and W⁹ are independently C or N; provided each of W⁵ and W⁹ is C when attached to R¹;
X and Y are independently an optionally substituted heteroaryl;
Z is an optionally substituted aryl or heteroaryl;
alternatively, Ring A together with Y may form a fused heteroaryl; or Y and Z together may form a fused heteroaryl; and
m and n are independently 0-2.

11. The compound of claim 10, wherein X is an optionally substituted pyrrolyl or imidazolyl.

12. The compound of claim 10, wherein Y is imidazolyl, triazolyl, or pyrazolyl; or Ring A together with Y form benzimidazolyl.

13. The compound of claim 1, wherein Z is an optionally substituted phenyl, pyridyl or furanyl; or Y and Z together form benzimidazolyl.

14. The compound of claim 1, wherein said compound is selected from the group consisting of

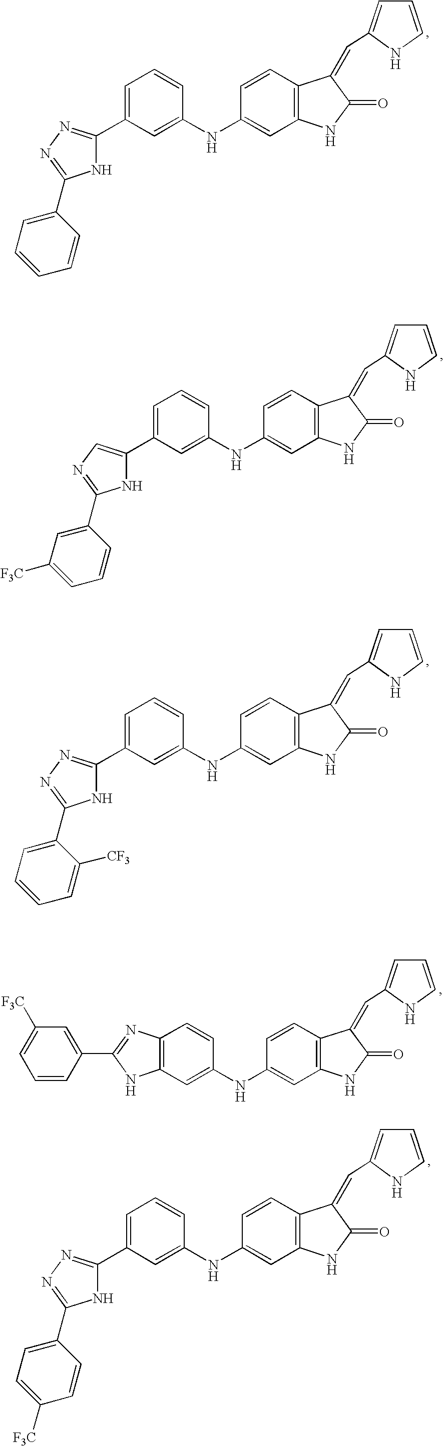

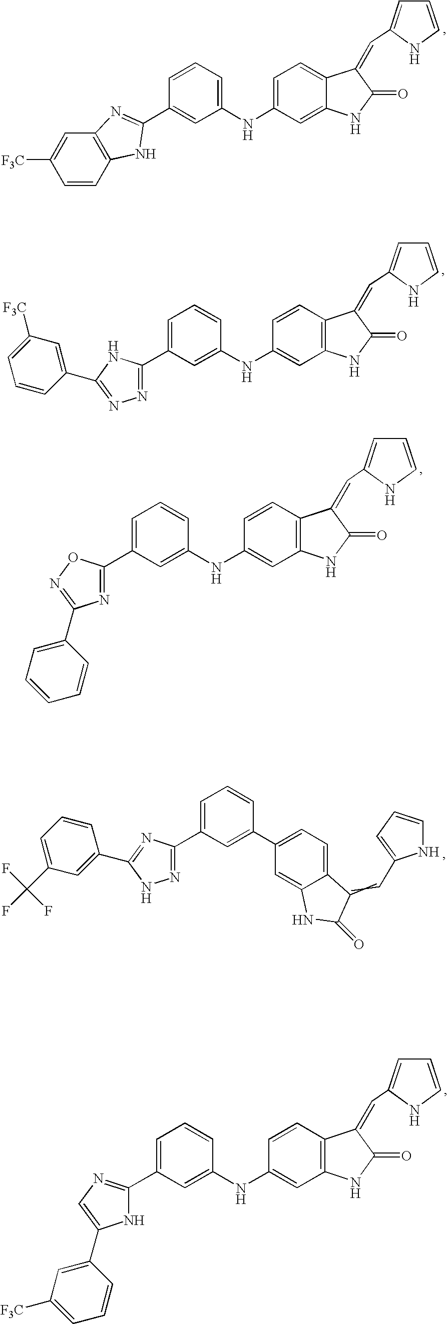

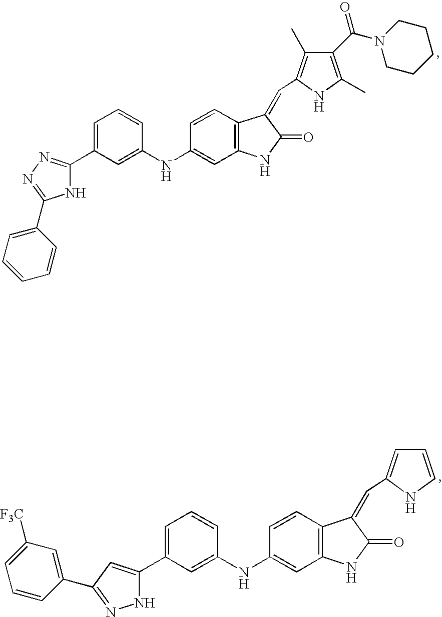

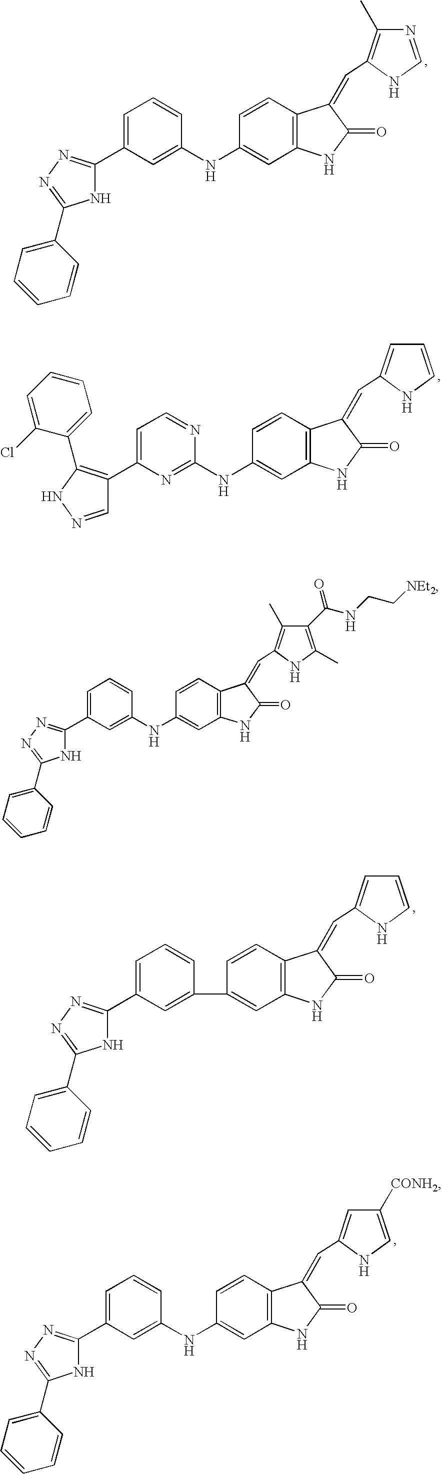

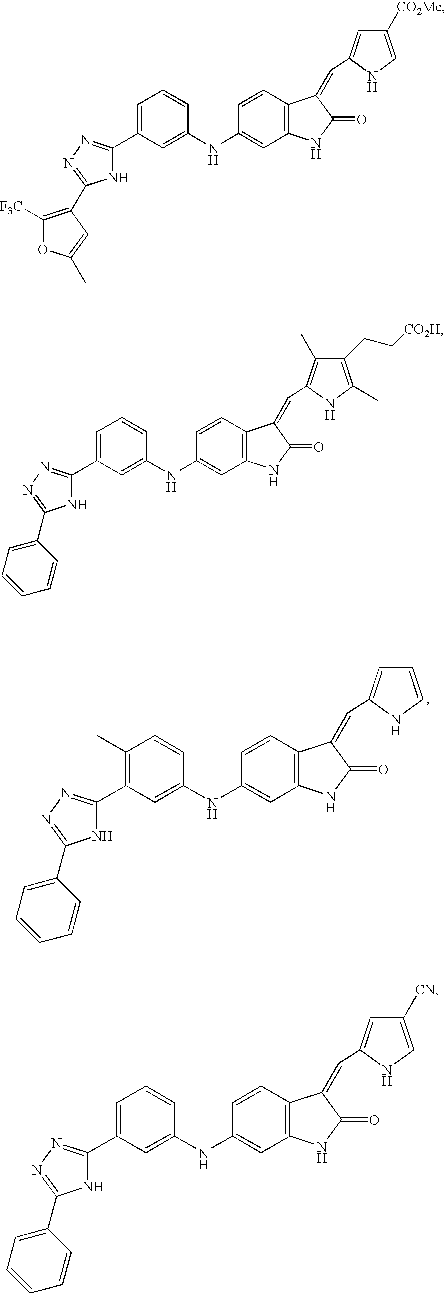

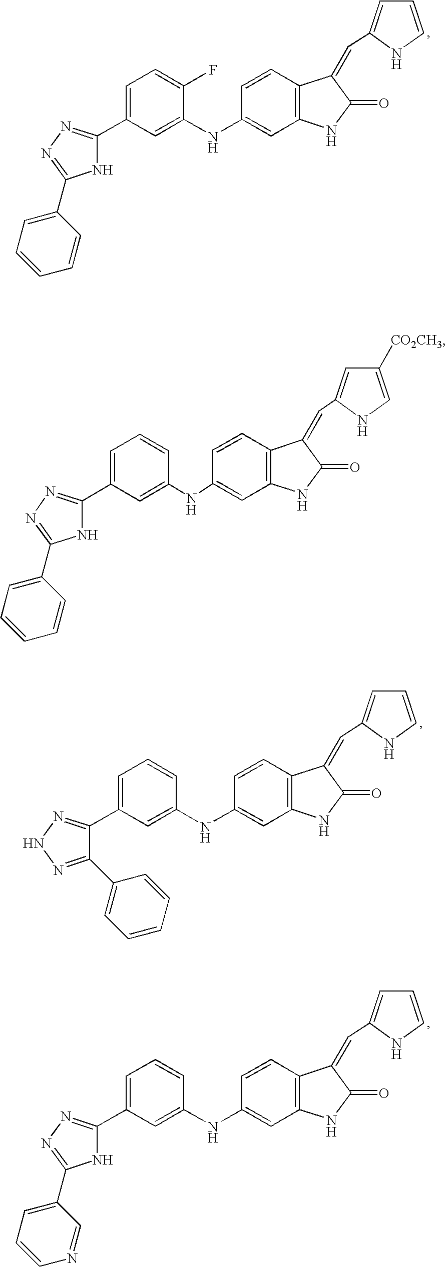

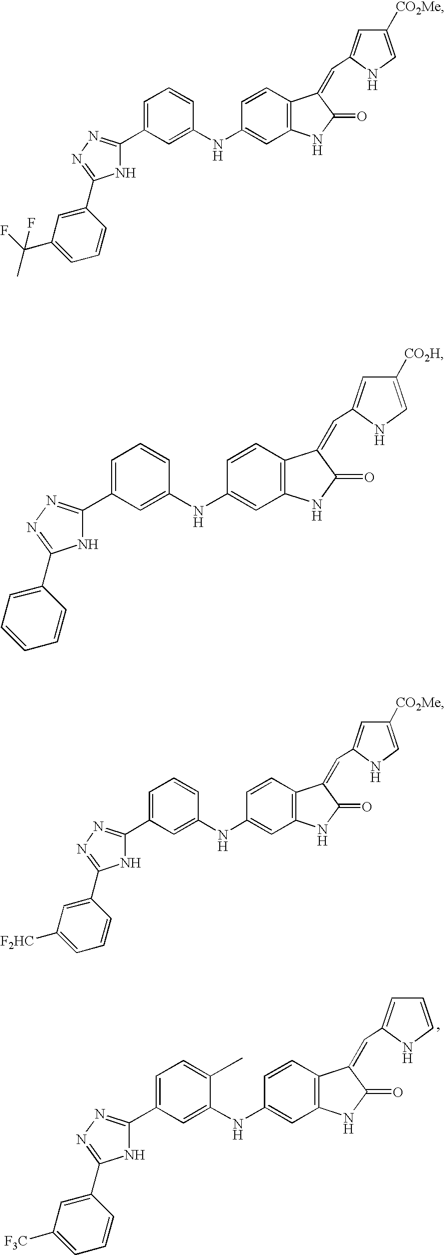

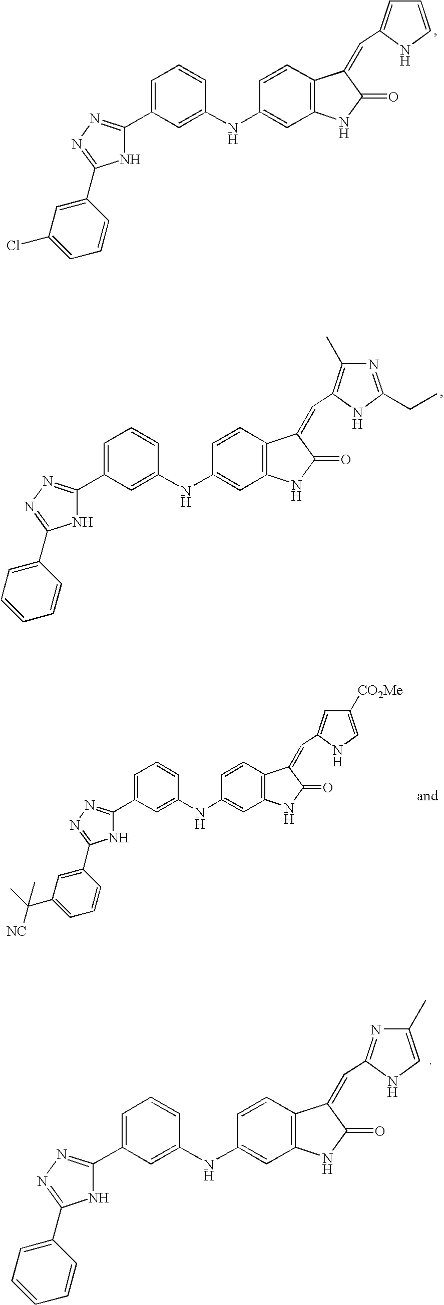

15. A pharmaceutical composition comprising a therapeutically effective amount of a compound of claim 1 and a pharmaceutically acceptable carrier.

16-20. (canceled)

21. A method for inhibiting Trk in a cell, comprising contacting the cell with an effective amount of a compound of claim 1 or a pharmaceutical composition thereof.

22. A method for treating a Trk-mediated condition in a subject in need thereof, comprising administering to the subject a therapeutically effective amount of a compound of claim 1 or a pharmaceutical composition thereof, and optionally in combination with a second therapeutic agent; wherein said condition is an autoimmune disease, a transplantation disease, an infectious disease or a cell proliferative disorder.

23. The method of claim 22, wherein said condition is a cell proliferative disorder, chronic pain, bone pain, abnormal angiogenesis, arthritis, diabetes, diabetic retinopathy, macular degeneration or psoriasis.

24. The method of claim 23, wherein said condition is a cell proliferative disorder selected from neuroblastoma and a tumor of the breast, prostate or pancreas.

25. A method for treating a cell proliferative disorder in a subject in need thereof, comprising administering to the subject a therapeutically effective amount of a compound of claim 1 or a pharmaceutical composition thereof, and optionally in combination with a second therapeutic agent; wherein said cell proliferative disorder is neuroblastoma, or a tumor of the breast, prostate or pancreas.

26. The method of claim 25, wherein said second therapeutic agent is a chemotherapeutic agent.
